SEQUENCE LISTING (14 sequences)

SEQ ID NO: 1 - DNA, 20 bp, Homo sapiens
atcaagaaggtggtgaagca

SEQ ID NO: 2 - DNA, 21 bp, Homo sapiens
gtcgctgttgaagtcagagga

SEQ ID NO: 3 - DNA, 19 bp, Homo sapiens
gagagtggtgcagcagcca

SEQ ID NO: 4 - DNA, 20 bp, Homo sapiens
ccactgttgcacagcagcat

SEQ ID NO: 5 - DNA, 23 bp, Homo sapiens
ttgggcatcatcttgctggttct

SEQ ID NO: 6 - DNA, 23 bp, Homo sapiens
tggctgacctgtttctcccactt

SEQ ID NO: 7 - DNA, 21 bp, Homo sapiens
gacttcgaaaagctgactaat

SEQ ID NO: 8 - DNA, 21 bp, Homo sapiens
ttggatgagttcatgtattgc

SEQ ID NO: 9 - DNA, 17 bp, Homo sapiens
ggagacttgcctggtaa

SEQ ID NO: 10 - DNA, 18 bp, Homo sapiens
gcatttgtggttgggtca

SEQ ID NO: 11 - DNA, 21 bp, Homo sapiens
ctgcagctctgtgtgaaggtg

SEQ ID NO: 12 - DNA, 25 bp, Homo sapiens
taagttctttagcactccttggcaa

SEQ ID NO: 13 - DNA, 20 bp, Homo sapiens
atggagttcgccagtcgaga

SEQ ID NO: 14 - DNA, 21 bp, Homo sapiens
cattgggcaacagagtctgct

(19) United States
(12) Patent Application Publication
(10) Pub. No.: US 2016/0281165 A1
(43) Pub. Date: Sep. 29, 2016

(54) METHODS FOR DIAGNOSING, SCREENING, IDENTIFYING, MONITORING, AND TREATING ADVERSE LOCAL TISSUE REACTIONS, WHICH LEAD TO FAILURE OF ORTHOPEDIC IMPLANTS

(71) Applicant: NEW YORK SOCIETY FOR THE RUPTURED AND CRIPPLED MAINTAINING THE HOSPITAL FOR SPECIAL SURGERY, New york, NY (US)

(72) Inventors: Steven Reyburn GOLDRING, New York, NY (US); Paul Edward PURDUE, Teaneck, NJ (US)

(21) Appl. No.: 15/030,142

(22) PCT Filed: Oct. 23, 2014
(86) PCT No.: PCT/US2014/061905

§ 371 Date: Apr. 18, 2016

Related U.S. Application Data
(60) Provisional application No. 61/895,496, filed on Oct. 25, 2013.

(51) Int. Cl. C12Q 1/68; G01N 33/573; G01N 33/68

(52) U.S. Cl. CPC C12Q 1/6883; G01N 33/6863; G01N 33/573; G01N 33/6866; G01N 33/6869; C12Q 2600/158; C12Q 2600/118; G01N 2333/5412; G01N 2333/5421; G01N 2333/522; G01N 2333/57; G01N 2333/926; G01N 2800/60

(57) ABSTRACT

This invention relates to field of screening and diagnosing adverse local tissue reactions or ALTR using proteins and genes that are elevated in patients suffering from ALTR, even those with no symptoms. The early diagnosis of the ALTR can lead to its treatment and thus, the prevention of implant failure caused by the ALTR. The elevated proteins and genes are also the basis for treatment for ALTR and provide targets for drug development and basic research.

### RELATED APPLICATION

The present application claims priority to U.S. patent application Ser. No. 61/895,496, filed Oct. 25, 2013, which is hereby incorporated by reference in its entirety.

### FIELD OF THE INVENTION

This invention relates to the field of diagnosing, screening for, and identifying adverse local tissue reactions or ALTR using proteins and genes that are elevated in patients suffering from ALTR, even those with no symptoms. The early diagnosis of the ALTR can lead to its treatment and thus, the prevention of implant failure caused by the ALTR.

The elevated proteins and genes are also the basis for treatment for ALTR and provide targets for drug development and basic research.

### BACKGROUND OF THE INVENTION

Total hip replacement (“THR”) or total hip arthroplasty (“THA”), as well as other joint replacements, is a highly successful treatment for degenerative arthritis, alleviating pain and restoring joint function in the vast majority of patients. However, it has been found that inflammatory reactions to implant-derived wear debris particles can lead to adverse local tissue reactions (“ALTR”) that ultimately lead to implant failure. The pathological features of the adverse tissue reactions, and the subsequent clinical course and outcomes are dependent upon the composition and biological properties of the wear products. Initial reports of ALTR after THR were attributable to polyethylene wear debris released from metal-on-polyethylene (“MoP”) implants. These tissue reactions were associated with the destruction of the peri-implant bone, a process identified as osteolysis, and accompanied by implant loosening and the need for revision surgery.

Modifications in bearing surface modularity and stem designs in total hip replacement were introduced in the past two decades with the goal of reducing the incidence of peri-implant osteolysis and aseptic loosening (Armstutz and Grigoris (1996); Srinivasan et al. (2012)). One of these modifications included the introduction of the metal-on-metal (“MoM”) bearing surfaces, which was combined with a metallic adapter sleeve for large heads in the early 2000s. The rationale for the revival of this bearing surface included: a reduction in volumetric wear and osteolysis compared to conventional metal-on-polyethylene bearings (“MoP”); decreased impingement throughout range of motion; and decreased rates of dislocation (Armstutz and Grigoris (1996)).

A second modification to increase modularity in THA was the introduction of the dual-modular neck (“DMN”). This provided surgeons with increased reconstructive options to potentially match each patient's anatomy and permit the use of a MoP or ceramic-on-polyethylene (“CoP”) bearing surface (Srinivasan et al. (2012)).

The unintended consequence of these implant modifications has been an increasing number of new interacting surfaces of different biomaterials, which has resulted in increased MoM and DMN implant failures due to a distinct type of cellular/tissue reaction, originally reported as aseptic lymphocyte dominated vasculitis-associated lesions (“ALVAL”), now collectively referred in the literature as adverse local tissue reactions (ALTR) or adverse reaction to metallic debris (“ARMD”) (Kop and Swarts (2009); Huber et al. (2009); Bosker et al. (2012); Chana et at (2012); Fabi et al. (2012); Gill et al. (2012); Meyer et al. (2012); Cooper et al. (2013); Werner et al. (2013)). Histological analyses of retrieved periprosthetic tissue have shown evidence of corrosion products, metallic debris generated by abrasion and/or surface fatigue, extensive soft tissue necrosis, combined macrophagic and lymphocytic infiltrate with variable plasmacytic and eosinophilic components, and vascular wall changes (Huber et at (2009); Davies and Willert (2005); Willert et al. (2005); Mahendra et al. (2009); Campbell et al. (2010); Natu et al. (2012); Grammatopoulos et al. (2013); Mittal et al. (2013)).

A comprehensive review describing features of periprosthetic inflammation to metallic wear debris has been addressed in a recent review article by Gallo et al. (2014). The constellation of pathologic findings observed in response to MoM implants was originally encompassed under the acronym ALVAL by Willert et al. (2005) to illustrate the unique lymphocytic component and probable vascular changes not seen in other typical modes of THA failure, such as osteolysis or infection. Taken together, these observations indicate that ALTRs associated with corrosion prone MoM and DMN devices represent an important subtype of implant ALTR (“corrosion-mediated ALTR”).

The tissue responses to metal corrosion products and the subsequent clinical course differ significantly from the tissue responses to polyethylene wear-induced tissue reactions in which implant failure is attributable primarily to osteolysis and loss of fixation. The recognition of metal corrosion product-induced tissue reactions thus expands the spectrum of ALTRs and indicates that there are distinct subtypes of ALTRs that are dependent upon the composition and biologic properties of the specific implant wear products. From a clinical perspective distinguishing between these specific ALTR subtypes is critical since each subtype is associated with distinct clinical patterns and mechanisms of failure that ultimately necessitate the need for implant removal and revision surgery.

Until recently, failure due to corrosion-mediated ALTR had predominantly been attributed and described for MoM bearing surfaces, but evidence of head-neck and neck-stem corrosion in DMN implants has been reported to result in corrosion-mediated ALTR, suggesting that this type of ALTR may impact more patients than originally suspected (Kop and Swarts (2009); Gill et al. (2012); Meyer et al. (2012); Cooper et al. (2013); Werner et al. (2013); Cooper et al, (2012); Fricka et al. (2012); Dyrkacz et al. (2013)).

Diagnosis and clinical options can be challenging for patients at risk of developing ALTR. Although unexplained pain is a frequent indicator of a potential corrosion-mediated ALTR, ALTR can develop in the absence of this symptom (Hart et al. (2012)). Imaging modalities, such as metal artifact reduction sequence magnetic resonance imaging (MARS MRI) have proved to be highly useful in identifying tissue reactions in THR patients at risk of ALTR, however, mass screenings via MARS MRI is expensive and impractical. Measurement of serum metal ions has proved to be of limited value as a marker of corrosion-mediated ALTR development, and to date there are no validated biomarkers for the early preclinical detection of osteolysis-associated ALTR associated with polyethylene wear products.

Therefore, there remains an unmet clinical need for better means of monitoring patients with implants at risk of ALTR. Importantly, this need extends beyond the ALTRs associated with metal corrosion products and includes ALTRs associated with polyethylene wear products, which presently represent the largest at risk population of patients with total joint replacements. Thus there is a critical need for the development of methods for monitoring the full spectrum of patients who are at risk for ALTRs, and for developing methods for discriminating between the distinct subtypes of ALTRs associated with specific implant wear products, since the clinical course and mechanisms of implant failure differ. Additionally a patient who has received an implant may not be aware of which type of implant they received and thus, which type of ALTR they are at risk for developing. The ultimate goal is to identify patients at risk for implant failure and to institute timely therapeutic interventions to prevent the need for implant revision not only in total hip replacement, but in other joints, such as knees and shoulders.

### SUMMARY OF THE INVENTION

The present invention provides a practical, feasible and inexpensive means for diagnosing, screening for, identifying, predicting and monitoring, as well as treating patients with, adverse local tissue reactions. The invention is based upon the finding that certain proteins and genes are elevated in patients with ALTR, prior to any noticeable symptoms. Since ALTR leads to implant failure, patients with ALTR, who often have no symptoms, can be identified by the methods of the present invention and appropriate medical intervention can be taken before implant failure and the need for revision surgery.

One embodiment of the present invention is a method and/or assay for diagnosing, screening for, identifying and/or predicting corrosion-mediated adverse local tissue reactions in a subject, comprising obtaining biological tissue and/or bodily fluid from the subject, purifying and/or isolating nucleic acid, including, but not limited to, mRNA, cDNA, and genomic DNA from the biological tissue and/or bodily fluid, measuring the level of the expression of one or more genes, and comparing the level or expression of the genes with a reference value for the level of expression of the same genes, wherein an increase in the level of expression of one or more genes from the subject as compared to the reference value would indicate the subject has corrosion-mediated ALTR.

Another embodiment of the invention is a method and/or assay for monitoring subjects for corrosion-mediated ALTR. This method and/or assay comprises obtaining biological tissue and/or bodily fluid from a subject, purifying and/or isolating nucleic acid, including, but not limited to, mRNA, cDNA and genomic DNA, from the biological tissue and/or bodily fluid, and measuring the level of expression of one or more genes, and comparing the level of expression of the genes with a reference value for the level of expression of the same genes, wherein an increase in the level of expression of one or more genes from the subject as compared to the reference value would indicate the subject should receive additional monitoring and testing for ALTR. Additional monitoring and testing would include, but is not limited to, repeating the method of the current invention at set intervals of time, and imaging via x-ray and/or MRI. The set intervals of time could be weekly, biweekly, monthly, bimonthly, quarterly, every six months, and yearly.

A method and/or assay used for monitoring for corrosion-mediated adverse local tissue reactions can also be used to monitor the effectiveness of a treatment for corrosion-mediated adverse local tissue reactions. This method and/or assay comprises obtaining biological tissue and/or bodily fluid from a subject, purifying and/or isolating nucleic acid, including, but not limited to, mRNA, cDNA and genomic DNA, from the biological tissue and/or bodily fluid, and measuring the level of expression of one or more genes, and comparing the level of expression of the genes with a reference value for the level of expression of the same genes, wherein no change or an increase in the level of expression of one or more the genes from the subject as compared to the reference value would indicate the treatment is not effective, and a decrease in the level of expression of one or more of the genes would indicate the treatment is effective. In this particular embodiment, the reference value could be the level of expression of the same genes from the subject prior to the treatment.

Another embodiment of the invention is a method of treating corrosion-mediated ALTR. This method comprises obtaining biological tissue and/or bodily fluid from a subject, purifying and/or isolating nucleic acid, including, but not limited to, mRNA, cDNA, and genomic DNA, from the biological tissue and/or bodily fluid, and measuring the level or expression of one or more genes, and comparing the level of expression of the genes with a reference value for the level of expression of the same genes, wherein an increase in the level of expression of one or more genes from the subject as compared to the reference value would indicate the subject has corrosion-mediated ALTR, and appropriate treatment can be prescribed.

In all of the above methods and assays for diagnosing, screening for, identifying, predicting, monitoring and treating corrosion-mediated ALTR, the genes of which the level of expression is measured include those listed in Table 4. In further embodiments, the genes include genes with chemokine activity and include, but are not limited to, CCL13, CCL2, CXCL13, CXCL9, CCL8, CCL19, CCL5, CCL4, CXCL12, CCL7, and CXCL10. In other embodiments, the genes include genes related to cellular metal ion homeostasis, and include, but are not limited to, CCL2, CCL19, FKBP1A, CCL5, CXCL12, CCL7, CD38, APP, CCL13, CXCL13, CXCR4, LCK, CCR2, and IL1B. In further embodiments, the genes include genes that are cytokines or cytokine receptors and include, but are not limited to, IL2RB, IL6, CCL2, IL21R, CXCL9, CCL19, CCL8, IL7R, CCL5, CXCL12, CCL4, CCL7, CXCL10, INHBA, CCL13, TNFSF10, CXCR4, CXCL13, CCR2, IL1B, IL13RA1, LTB, and CD27. In other embodiments, the genes include those listed in Table 2. In preferred embodiments, the genes are chosen from the group consisting of CXCL9, CXCL10, IFNγ, IL6 and IL8.

Determining the expression of any of the genes can be done by any method known in the art, including, but not limited to, microarrays; Southern blots; Northern blots; dot blots; primer extension; nuclease protection; subtractive hybridization and isolation of non-duplexed molecules using, for example, hydroxyapatite; solution hybridization; filter hybridization; amplification techniques such as RT-PCR and other PCR-related techniques, such as PCR with melting curve analysis, and PCR with mass spectrometry; fingerprinting, such as with restriction endonucleases; the use of structure specific endonucleases; and RNA sequencing. mRNA expression can also be analyzed using mass spectrometry techniques (e.g., MALDI or SELDI), liquid chromatography, and capillary gel electrophoresis. Any additional method known in the art or developed in the future can be used to measure or determine the expression of the genes and/or measure or detect nucleic acids in a sample. Preferred methods are microarrays and RNA sequencing.

The level of expression of the genes in the subject can be compared to a reference value of the level of expression of the same genes in a control. This reference value can be fold change differences of specific RNA in comparison to housekeeping genes.

In one embodiment, the level of expression of the genes in the subject is compared to a reference value of the level of expression of the same genes from the subject prior to, at the time of, or shortly after, receiving the implant.

Another embodiment of the present invention is a method and/or assay for diagnosing, screening for, identifying and/or predicting corrosion-mediated adverse local tissue reactions in a subject, comprising obtaining biological tissue and/or bodily fluid from the subject, purifying and/or isolating protein from the biological tissue and/or bodily fluid, and measuring the amount of protein or proteins and comparing the amount with a reference value for the amount of the same protein or proteins, wherein an increase in the amount of protein or proteins from the subject as compared to the reference value would indicate the subject has corrosion-mediated ALTR. In a preferred embodiment, the protein is chosen from the group consisting of CXCL9, CXCL10, IFNγ, IL6 and IL8. In a further embodiment, the protein is chosen from the proteins encoded by any of the genes listed in Tables 2 and 4.

Another embodiment of the invention is a method and/or assay for monitoring subjects for corrosion-mediated ALTR. This method and/or assay comprises obtaining biological tissue and/or bodily fluid from a subject, purifying and/or isolating protein from the biological tissue and/or bodily fluid, and measuring the amount of protein or proteins and comparing the amount with a reference value for the amount of the same protein or proteins, wherein an increase in the amount of protein or proteins from the subject as compared to the reference value would indicate the subject should receive additional monitoring and testing for ALTR. In a preferred embodiment, the protein is chosen from the group consisting of CXCL9, CXCL10, IFNγ, IL6 and IL8. In a further embodiment, the protein is chosen from the proteins encoded by any of the genes listed in Tables 2 and 4. Additional monitoring and testing would include, but is not limited to, repeating the method of the current invention at set intervals of time, and imaging via x-ray and/or MRI. The set intervals of time could be weekly, biweekly, monthly, bimonthly, quarterly, every six months, and yearly.

A method and/or assay used for monitoring for corrosion-mediated adverse local tissue reactions can also be used to monitor the effectiveness of a treatment for corrosion-mediated adverse local tissue reactions. This method and/or assay comprises obtaining biological tissue and/or bodily fluid from a subject, purifying and/or isolating protein from the biological tissue and/or bodily fluid, and measuring the amount of protein or proteins and comparing the amount with a reference value for the amount of the same protein or proteins, wherein no change or an increase in the amount of protein or proteins from the subject as compared to the reference value would indicate the treatment is not effective, and a decrease in the amount of protein or proteins from the subject as compared to the reference value would indicate the treatment is effective. In a preferred embodiment, the protein is chosen from the group consisting of CXCL9, CXCL10, IFNγ, IL6 and IL8. In a further embodiment, the protein is chosen from the proteins encoded by any of the genes listed in Tables 2 and 4. In this particular embodiment, the reference value could be the level of the same protein or proteins from the subject prior to the treatment.

Another embodiment of the invention is a method of treating corrosion-mediated ALTR. This method comprises obtaining biological tissue and/or bodily fluid from a subject, purifying and/or isolating protein from the biological tissue and/or fluid, and measuring the amount of protein or proteins and comparing the amount with a reference value for the amount of the same protein or proteins, wherein an increase in the amount of protein from the subject as compared to the reference value would indicate the subject has corrosion-mediated ALTR. In a preferred embodiment, the protein is chosen from the group consisting of CXCL9, CXCL10, IFNγ, IL6 and IL8. In a further embodiment, the protein is chosen from the proteins encoded by any of the genes listed in Tables 2 and 4. If the amount of one or more protein is increased in the subject appropriate treatment can be prescribed.

Detection of the levels of proteins can be accomplished by any method known in the art, including methods which result in qualitative results, such as ones where the existence of the protein can be visualized, either by the naked eye or by other means, and/or quantitative results. Such methods would include, but are not limited to, quantitative Western blots, immunoblots, quantitative mass spectrometry, enzyme-linked immunosorbent assays (ELISAs), radioimmunoassays (RIA), immunoradiometric assays (IRMA), and immunoenzymatic assays (IEMA) and sandwich assays using monoclonal and polyclonal antibodies. The amounts of protein, chosen from the group consisting of CXCL9, CXCL10, IFNγ, IL6 and IL8, as found in the fluid and tissue of patients suffering from corrosion-mediated ALTR and patients suffering from osteolysis-associated ALTR, are found in Table 1.

Again in one embodiment, the amount of the protein or proteins is compared to a reference value of a measurement of the amount of the same protein or proteins from the subject prior to, at the time of; or shortly after, receiving the implant.

Any subject who has received an implant is at risk for ALTR and can benefit from these screening, diagnosing and monitoring methods, as ALTR can develop in less than a year after receiving an implant and often without symptoms. There are factors that may increase a subject's risk of ALTR, including the time of implant, pain, type of implant, sex, and metal ion levels. However, because the present invention provides a feasible and inexpensive means for diagnosing, screening for, identifying, predicting and/or monitoring adverse local tissue reaction, all subjects who have received an implant should be considered a candidate for the methods. These methods and/or assays can be performed at any time from about six months after the subject received the implant.

Again because patients may frequently not know what type of ALTR for which they may be at risk, corrosion-mediated and/or osteolysis-associated, the present invention also includes methods and assays for diagnosing, screening for, identifying, predicting, monitoring, and treating both corrosion-mediated and/or osteolysis-associated adverse local tissue reactions in a given subject. These methods and assays would include additionally detecting nucleic acids associated with osteolysis-associated ALTR, CHIT1 and CCL18, as well as the proteins themselves, along with the nucleic acids and proteins associated with corrosion-mediated ALTR, using the techniques outlined above. Alternatively, the genes listed in Table 3 could be used in methods and/or assays for diagnosing, screening for, identifying, predicting, monitoring, and treating osteolysis-associated ALTR.

If a subject is found to have corrosion-mediated ALTR, the proper treatment and/or further testing can be determined. Alternatively, if a subject is found to have osteolysis-mediated ALTR, the proper treatment and/or further testing can be determined.

A method of treating a subject with corrosion-mediated ALTR could comprise administering to a subject in need thereof a therapeutically effective amount of an agent that decreases, prevents or blocks the expression of the CXCL9 and/or CXCL10 and/or IFNγ and/or IL6 and/or IL8 gene, or one of the other genes listed in Tables 2 and 4, or an agent that decreases, prevents or blocks the activation, amount and/or activity of CXCL9 and/or CXCL10 and/or IFNγ and/or IL6 and/or IL8, or of a protein encoded by one of the genes listed in Tables 2 and 4.

A method of treating a subject with osteolysis-associated ALTR could comprise administering to a subject in need thereof a therapeutically effective amount of an agent that decreases, prevents or blocks the expression of the CHIT1 and/or CCL18 gene, or one of the other genes listed in Table 3, or an agent that decreases, prevents or blocks the activation, amount and/or activity of CHIT1 and/or CCL18, or of a protein encoded by one of the genes listed in Table 3.

The present invention also provides for methods and tools for drug design, testing of agents, and tools for basic research into the causes and etiology of ALTR.

One embodiment is a method and/or assay for screening and/or identifying a test agent for the prevention and/or treatment of corrosion-mediated ALTR, comprising contacting or incubating the test agent with a protein chosen from the group consisting of CXCL9, CXCL10, IFNγ, IL6 and IL8, or a protein encoded by any of the genes listed in Tables 2 and 4, and detecting the presence of a complex between the test agent and the protein, wherein if a complex between the test agent and the protein is detected, the test agent is identified as a prevention and/or treatment for corrosion-mediated ALTR.

A further embodiment is a method and/or assay for screening and/or identifying a test agent for the prevention and/or treatment of corrosion-mediated ALTR, comprising contacting or incubating the test agent with a protein chosen from the group consisting of CXCL9, CXCL10, IFNγ, IL6 and IL8, or a protein encoded by any of the genes listed in Tables 2 and 4, and a known ligand of the protein, and detecting the presence of a complex between the test agent and the ligand, wherein if a complex between the test agent and the ligand is detected, the test agent is identified as a prevention and/or treatment for corrosion-mediated ALTR.

Another embodiment of the present invention is a method and/or assay for screening and/or identifying a test agent for the prevention and/or treatment of corrosion-mediated ALTR, comprising contacting or incubating the test agent with a protein chosen from the group consisting of CXCL9, CXCL10, IFNγ, IL6 and IL8, or a protein encoded by any of the genes listed in Tables 2 and 4, and a known antibody of the protein, and detecting the presence and quantity of unbound antibody, wherein the presence of the unbound antibody indicates that the test agent is binding to the protein, and the test agent is identified as a prevention and/or treatment for corrosion-mediated ALTR.

These methods and assays can be performed with the polypeptides and test agents, and ligands and antibodies, if applicable, free in solution, or affixed to a solid support. The polypeptides and antibodies may be labeled by any method known in the art.

High throughput screening can also be used to screen the test agents. Small peptides or molecules can be synthesized and bound to a surface and contacted with the polypeptides encoded by the gene signature transcripts, and washed. The bound peptide is visualized and detected by methods known in the art.

A further embodiment of the present invention is a method and/or assay for screening and/or identifying a test agent for the prevention and/or treatment of corrosion-mediated ALTR comprising contacting or incubating a test agent to a nucleotide encoding the protein chosen from the group consisting of CXCL9, CXCL10, IFNγ, IL6 and IL8, or any one of the genes listed in Tables 2 and 4 and determining if the test agent binds to the nucleotide, wherein if the test agent binds to the nucleotide, the test agent is identified as a prevention and/or treatment for corrosion-mediated ALTR.

A further embodiment of the present invention is a method and/or assay for screening and/or identifying a test agent for the prevention and/or treatment of corrosion-mediated ALTR comprising contacting or incubating a test agent with a nucleotide encoding the protein chosen from the group consisting of CXCL9, CXCL10, IFNγ, IL6 and IL8, or any one of the genes listed in Tables 2 and 4 which expresses a measurable phenotype, and measuring the phenotype before and after contact or incubation with the test agent, wherein if the expression of the measurable phenotype is decreased after the contact or incubation with the test agent, the test agent is identified as a prevention and/or treatment for corrosion-mediated ALTR.

The measurable phenotype can be one that is native to the gene or one that is artificially linked, such as a reporter gene.

A further embodiment of the present invention is a method and/or assay for screening and/or identifying a test agent for the prevention and/or treatment of corrosion-mediated ALTR, comprising transforming a host cell with a gene construct comprising a nucleotide encoding the protein chosen from the group consisting of CXCL9, CXCL10, IFNγ, IL6 and IL8, or any one of the genes listed in Tables 2 and 4, detecting the expression of the gene in the host cell, contacting or incubating the test agent with the host cell, and detecting the expression of the gene from the host cell after contact with the test agent, wherein if the expression of the gene is reduced or decreased after contact or incubation with the test agent, the test agent is identified as a prevention and/or treatment for corrosion-mediated ALTR.

All of the above methods and assays for screening and/or identifying preventative and therapeutic agents for corrosion-mediated ALTR can also be performed using CHIT1 and/or CCL18 proteins and genes encoding CHIT1 and/or CCL18, or the genes listed in Table 3 and proteins encoded by the genes listed in Table 3, to screen for and/or identify preventative and/or therapeutic agents for osteolysis-associated ALTR.

The present invention also provides methods for using target genes or proteins for drug development and basic research regarding ALTR.

The present invention also includes kits.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 is a cytokine and chemokine antibody array of synovial fluid from patients with corrosion-mediated ALTR and those with osteolysis-associated ALTR.

FIG. 2 shows graphs of the results of ELISA on synovial fluid from patients with osteolysis-associated ALTR (“osteolysis”), corrosion-mediated ALTR from the DMN group (“ALTR-DMN”), and corrosion-mediated ALTR from the MoM group (“ALTR-MoM”). Levels of IP10 (CXCL10), MIG (CXCL9), IL6, IL8, IFN gamma and CHIT1 are shown for the three groups.

FIG. 3A is a cytokine and chemokine antibody array of serum from patients with corrosion-mediated ALTR and those with osteolysis-associated ALTR. FIG. 3B shows graphs of the results of ELISA on serum from patients with osteolysis-associated ALTR (“Osteolysis”), corrosion-mediated ALTR from the DMN group (“ALTR-DMN”), and corrosion-mediated ALTR from the MoM group (“ALTR-MoM”). Levels of IP10 (CXCL10) and MIG (CXCL9) are shown for the three groups.

FIG. 4A is a gene expression profile of tissue from patients with corrosion-mediated ATLR in the DNM group and patients with osteolysis-associated ALTR. Each column represents a patient, with the first eight columns on the left hand side being patients with corrosion-mediated ALTR and the eight columns on the right hand side being patient with osteolysis-associated ALTR. Each row represents an expressed gene. FIG. 4B are graphs of the results of reverse transcription-polymerase chain reaction for MIG (CXCL9), IFNγ, IL8, IL6, CD3 and CHIT1 in patients with corrosion-mediated ALTR from the DMN group (“ALTR (DMN)”), corrosion-mediated ALTR from the MoM group (“ALTR (MoM)”), and patients with osteolysis-associated ALTR (“osteolysis”). Each bar represents one patient and there are two patients from each group shown.

### DETAILED DESCRIPTION OF THE INVENTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. In some embodiments of the present invention, the “patient” is one who received a THA or an implant at another joint such as a knee or shoulder, and/or may be suffering from ALTR, suspected of suffering from ALTR, or at risk for developing ALTR.

The terms “implant”, “device”, and “prosthetic”, are used interchangeably throughout this application and means any material inserted or grafted into the body that maintains support and tissue contour.

The terms “total hip replacement” or “THR” or “total hip arthroplasty” or “THA” as used herein mean the implantation of an implant or device in a subject to replace an existing diseased or injured hip.

The term “joint” is defined as the area where two bones are attached for the purpose of permitting body parts to move and comprise a variety of musculoskeletal tissue, including cartilage, tendons, and ligament.

The terms “metal-on-polyethylene” or “MoP” are used interchangeably herein and refer to a type of implant for THA containing a metal stem, neck, and head, and a polyethylene liner and shell (bearing surface). The terms “ceramic-on-polyethylene” or “CoP” are used interchangeably herein and refer to a type of implant for THA containing a ceramic head, a metal stem, and a polyethylene liner.

The terms “metal-on-metal” or “MoM” are used interchangeably herein and refer to a type of implant for THA containing a metal stem, neck, head, liner, and shell (bearing surface).

The terms “modular neck” or “dual-modular neck” or “DMN” are used interchangeably herein and refer to a type of implant for THA containing a modular neck, used to customize the implant to the subject's anatomy, and a MoP or CoP bearing surface.

The terms “adverse local tissue reactions” or “ALTR” are used interchangeably herein and refer to the periprosthetic local soft tissue and/or bone inflammation and tissue injury, composed of an inflammatory cell infiltrate, with or without extensive soft tissue necrosis, and vascular changes.

The terms “corrosion-mediated adverse local tissue reactions” or “corrosion-mediated ALTR” are used interchangeably herein and mean the subtype of ALTR associated with corrosion prone implants, such as MoM and DMN hip implants.

The term “osteolysis” as used herein means bone loss.

The terms “osteolysis-associated adverse local tissue reactions” or “osteolysis-associated ALTR” are used interchangeably herein and mean the subtype of ALTR associated with bone loss, and polyethylene implant wear products.

The terms “screen” and “screening” and the like as used herein means to test a subject or patient to determine if they have a particular illness or disease, or a particular manifestation of an illness or disease. The term also means to test an agent to determine if it has a particular action or efficacy.

The terms “identification”, “identify”, “identifying” and the like as used herein means to recognize a disease state or a clinical manifestation or severity of a disease state in a subject or patient. The term also is used in relation to test agents and their ability to have a particular action or efficacy.

The terms “diagnosis”, “diagnose”, diagnosing” and the like as used herein means to determine what physical disease or illness a subject or patient has.

The terms “prediction”, “predict”, “predicting” and the like as used herein means to tell in advance based upon special knowledge.

The term “reference value” as used herein means an amount or a quantity of a particular protein or nucleic acid or level of gene expression in a sample from a control, or the amount or quantity of a particular protein or nucleic acid or level of gene expression in a sample from the subject prior to, at the time of, or shortly after, receiving the implant, or prior to receiving treatment, where the sample is taken from the same bodily fluid or biological tissue and similarly processed. The “reference value” can also include a predetermined value such as a value published in a reference or otherwise known in the art.

The term “control” as used in this application is a subject who is not suffering from ALTR or in some embodiments not suffering from the same subtype of ALTR, i.e., corrosion-mediated versus osteolysis-associated.

The term “similarly processed” as used herein would mean a sample is obtained and processed from bodily fluid or biological tissue using the same or similar protocol.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “agent” as used herein means a substance that produces or is capable of producing an effect and would include, but is not limited to, chemicals, pharmaceuticals, biologics, small organic molecules, antibodies, nucleic acids, peptides, and proteins.

The phrase “therapeutically effective amount” is used herein to mean an amount sufficient to cause an improvement in a clinically significant condition in the subject, or delays or minimizes or mitigates one or more symptoms associated with the disease, or results in a desired beneficial change of physiology in the subject.

The phrase “in need thereof” is used herein to mean a subject who has either developed ALTR or is at risk for developing ALTR.

The phrase “at risk for developing ALTR” as used herein means a subject who has received an implant to a joint, including, but not limited to, a hip, knee or shoulder.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

The term “purified” and the like as used herein refers to material that has been isolated under conditions that reduce or eliminate unrelated materials, i.e., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The terms “expression profile” or “gene expression profile” refers to any description or measurement of one or more of the genes that are expressed by a cell, tissue, or organism under or in response to a particular condition. Expression profiles can identify genes that are up-regulated, down-regulated, or unaffected under particular conditions. Gene expression can be detected at the nucleic acid level or at the protein level. The expression profiling at the nucleic acid level can be accomplished using any available technology to measure gene transcript levels. For example, the method could employ in situ hybridization, Northern hybridization or hybridization to a nucleic acid microarray, such as an oligonucleotide microarray, or a cDNA microarray. Alternatively, the method could employ reverse transcriptase-polymerase chain reaction (RT-PCR) such as fluorescent dye-based quantitative real time PCR (TaqMan® PCR), In the Examples section provided below, nucleic acid expression profiles were obtained using Affymetrix GeneChip® oligonucleotide microarrays. The expression profiling at the protein level can be accomplished using any available technology to measure protein levels, e.g., using peptide-specific capture agent arrays.

The terms “gene”, “gene transcript”, and “transcript” are used somewhat interchangeable in the application. The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. “Transcript” or “gene transcript” is a sequence of RNA produced by transcription of a particular gene. Thus, the expression of the gene can be measured via the transcript.

The term “antisense DNA” is the non-coding strand complementary to the coding strand in double-stranded DNA.

The term “genomic DNA” as used herein means all DNA from a subject including coding and non-coding DNA, and DNA contained in introns and exons.

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include, but are not limited to, plasmids, phages, and viruses.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described herein.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

“Nucleic acid” refers to deoxyribonucleotides or ribonucleotides and polymers thereof in either single- or double-stranded form. The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′- non-coding regions, and the like. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. The nucleic acids may also be modified by many means known in the art.

The term “polypeptide” as used herein means a compound of two or more amino acids linked by a peptide bond. “Polypeptide” is used herein interchangeably with the term “protein.”

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

Biomarkers of Osteolysis-Associated Adverse Local Tissue Reactions in Patients with Implants

Osteolysis and periprosthetic loosening are considered major long term complications of joint replacement, often resulting in the need for revision surgery. Development of periprosthetic osteolysis is characterized by wear debris accumulation along the periprosthetic membrane and phagocytosis by macrophages (Schmalzried et al. (1992)). This clinical subtype of ALTR characteristically is associated with host responses to polyethylene implant wear products. The failure mechanism most often is attributable to osteolysis and loss of fixation requiring the need for revision surgery.

In a previous reports by the inventors, the hypothesis that osteolysis-associated ALTR is characterized by an alternative macrophage activation pathway within the periprosthetic tissue distinguished by cytokine and chemokines but not pro-inflammatory mediators was tested (Koulouvaris et al. (2007) and Lovy et al. (2009), both herein incorporated by reference in their entirety).

After the comparison of key mediators of macrophage activation, osteoclast precursor recruitment, and activation, and osteogenesis between patients with osteolysis-associated ALTR and controls, CHIT1 and CCL18 mRNA, both markers of alternatively activated macrophages, were found to be elevated in the tissues of patients with osteolysis-associated ALTR, with fold differences of 66.3 and 2.9, between patients with osteolysis-associated ALTR and controls, respectively. Additionally, CHIT1 and CCL18 protein levels were increased in the serum of patients with osteolysis-associated ALTR. Patients with osteolysis-associated ALTR had an average of 407.9 nmol/ml/hr of CHIT1 as compared to an average of 92 in the controls, and 78.9 ng/ml of CCL18 as compared to an average of 41.9 in the controls.

Thus, CHIT1 and CCL18 are valid biomarkers for osteolysis-associated ALTR both at the nucleic acid and protein level.

Corrosion-Mediated Adverse Local Tissue Reactions in Patients with Implants

Corrosion-mediated adverse local tissue reaction or ALTR is a locally destructive pathology associated with certain types of total hip replacement implants and other joint implants. Corrosion-mediated ALTR is characterized by periprosthetic soft tissue inflammation composed of mixed inflammatory cell infiltrate, extensive soft tissue necrosis, and vascular changes. Failure of implants from corrosion-mediated ALTR has been indicated as a risk factor for revision surgery.

Despite recent significant advances in defining the spectrum of histopathological features associated with corrosion-mediated ALTR, a mechanistic understanding of the cellular and molecular factors underlying this reaction remains to be elucidated. From a biomaterials standpoint, attention has been focused on corrosion products as putative initiating factors or mediators of the cellular responses. Although somewhat poorly defined, these wear products can be identified and distinguished from classical metallic debris by virtue of a characteristic green appearance. Corrosion products can be seen associated with the surfaces of failed devices, as well as within the peri-implant tissues, both as large extracellular aggregates and smaller intracellular inclusions, and dual X-ray absorption spectroscopy studies have indicated that a major component of these particles is implant derived chromium orthophosphate. Major unanswered questions include how these corrosion products are generated, how they become infiltrated throughout the tissues and how they contribute to the destructive reactions seen in corrosion-mediated ALTR.

In a previous report by the inventors, the histologic and immunohistochemical features of corrosion-mediated ALTR in patients with MoM and DMN implants was studied and classified (Perino et al. (2014), herein incorporated by reference in its entirety). In this study, histological evidence showed morphologic features in corrosion-mediated ALTR with an early phase of cellular activation and proliferation seen in neo-synovial reaction to other particulate implant materials followed by a distinctive sequence of cellular and tissue reactions leading to the formation of a soft tissue necrosis.

This study also demonstrated an association between the presence of extra-cellular and intra-cellular corrosion products in the periprosthetic tissue with the presence of interstitial and perivascular lymphocytic infiltrate. Large aggregates of corrosion products were seen in the soft tissues of the subjects in the study. Both groups of subjects, those with MoM and DMN implants, had particle laden macrophages, although with slight differences in appearance. The association suggests that corrosion particle laden macrophages are instrumental in the formation of the lymphocytic infiltrate, although free particulate material can also significantly contribute to the response.

Three distinct histologic patterns were identified at light microscopy in this reported study: 1) a predominantly macrophagic pattern with absent or minimal lymphocytic response; 2) mixed inflammatory pattern, macrophagic and lymphocytic with variable presence of plasma cells, eosinophils, and mast cells; and 3) granulomatous pattern, predominant or associated with the inflammatory pattern. The predominantly macrophagic group represented a group of patients with an adverse soft tissue reaction resulting in implant failure with minimal lymphocytic activation (Campbell et al. (2010); Grammatopoulos et al. (2013)). The mixed inflammatory pattern was subdivided into those with (A) or without (B) germinal centers. The third group had prominent formation of sarcoidosis-like granulomas in the presence of a mixed or macrophagic infiltrate, and this may represent a subset of patients with particular macrophage characteristics (Mahendra et al. (2009); Natu et al. (2012)).

The study also reported immunohistochemistry results that showed a predominant T lymphocytic response with a variable B cell component with the formation in some cases of perivascular germinal centers and tall endothelial cell venules. The analysis of the T cell population pointed towards a mixed pattern with predominant GATA3 positivity (Th2 lymphocyts) but also substantial T-bet and FOXP3 expressing lymphocytes, representing Th1 and Treg subgroups respectively. These findings were also associated with the presence of macrophages strongly positive for CD163, a marker of M2 macrophages, a subset frequently correlated with Th2 cytokines. There was also a finding of a variable number of CD117 positive mast cells.

Perhaps one of the most important conclusions of this study was the need for a practical clinical method for diagnosing corrosion-mediated ALTR. The study reported that the use of the proposed ALVAL score by Campbell et al. would be of limited clinical value in predicting a course or outcome of corrosion-mediated ALTR because the score would be an indication of developmental stage of the adverse reaction and not a grade of its biological severity, and pointed to a need for a method for monitoring and diagnosing corrosion-mediated ALTR.

Use of Biomarkers to Diagnose, Screen for, Identify, Predict, Monitor or Treat Adverse Local Tissue Reactions Associated with Implant Wear-Induced Products

Provided herein are a characterization of the molecular features of the subtypes of ALTRs that are associated with the distinct implant wear products. Dysregulated proteins and gene expression profiles have been identified that permit preclinical detection of the ALTR and importantly can discriminate between the distinct subtypes of ALTR associated with polyethylene wear and metal corrosion wear products. These studies not only provide the first clues as to the affected signaling networks associated with ALTR, but also identify novel pathologically up-regulated components that have the potential to serve as disease biomarkers and/or therapeutic targets.

By using these biomarkers, alone or preferably in conjunction, important predictions and determinations can be made regarding the course of a patient's progression who has received a joint implant. Because these biomarkers, which are in the form of nucleic acids and proteins, can obtained from bodily fluid or biological tissue by non-invasive or minimally invasive, and inexpensive techniques, (e.g., as opposed to expensive and sometimes uncomfortable imaging techniques), the methods and assays of the current invention allow for inexpensive, practical and non- or minimally, invasive diagnostic techniques that can easily be performed on any subject who has received an implant to a joint, even if the implant has been recent and/or the subject is experiencing no symptoms. In that manner, valuable insight into the clinical manifestations of the disease can be utilized in both choice of therapy as well as the determination for the amount and timing needed for monitoring by a health care provider, often time prior to any noticeable symptoms.

In a preferred embodiment, the serum is tested for one or more of these biomarkers, as serum is the most convenient fluid or tissue for later testing.

In one embodiment, a subject receiving an implant would be tested for one or more of these biomarkers prior to, during, or a short time after, the implantation of the implant. In this manner, there would be a reference value of a measurement of the biomarker in a disease free state of the subject to be used for comparison for later testing.

In another embodiment, the reference value can be from a known value of the biomarker in a control. As defined, a control can be a subject not suffering from ALTR. A reference value can also be the amount of the protein or level of gene expression in a control with one subtype of ALTR versus the other. For example, the mean amounts of CXCL9, CXCL10, IL6 and IL8 proteins in synovial fluid and serum for patients with corrosion-mediated ALTR and osteolysis-associated ALTR are listed in Table 1. Additionally, the fold change difference in gene expression of CXCL9 and CXCL10 in patients with corrosion-mediated ALTR as compared to patients with osteolysis-associated ALTR is found in Table 2.

The reference value can also be ascertained from a published reference.

The disclosed methods and assays can be used to diagnose, screen for, identify, and/or predict ALTR in any subject with an implant, whether or not suffering symptoms. When ALTR is positively diagnosed, the subject can then be treated accordingly.

The disclosed methods and assays can also be used to monitor a subject who has had an implant for ALTR whether or not suffering symptoms. When used to monitor the subject, an increase in the biomarker may lead to treatment, but also may lead to further testing such as imaging via MRI or x-ray. An increase may also lead to a determination for the amount and timing needed for monitoring by a health care provider in the form of additional testing for the biomarkers, at more frequent intervals or for testing of additional biomarkers.

The disclosed methods and assays can also be used to monitor a subject who is being treated for ALTR to determine if the treatment is effective or not. When used in this manner, it can be determined whether the treatment has successfully treated the ALTR and can be stopped or continued, or whether the treatment has been ineffective, and what additional or alternative treatment can be performed or administered to the subject.

The disclosed methods and assays can also be used to treat a subject who has had an implant. In this embodiment, a subject who has tested positive for ALTR due to an increase level of one or more of the biomarkers can be treated using one or more of the following treatments set forth below.

The methods and the assays of the current invention can also be used to prevent implant failure that is caused by ALTR.

The most common type of implant at risk for developing corrosion-mediated ALTR are MoM and DMN type implants used for total hip replacements. Osteolysis-associated ALTR associated with polyethylene wear products represent an additional distinct subset of patients. There is a critical need for the development of biomarkers that can identify the specific subtype of ALTR, since the clinical presentation, course and potential therapeutic intervention may differ. Additionally, patients with other implants used in hips and implants at other joints, including, but not limited to knees and shoulders, can also benefit from the use of the biomarkers for ALTR.

Genes Correlated with ALTR

As shown in the examples, there is an up-regulation of certain genes associated with corrosion-mediated ALTR. Thus, these genes can be specifically used to screen for, identify, diagnose, predict, monitor and/or treat corrosion-mediated ALTR in a subject who has had a THA or another joint implant, or who is at risk for ALTR. These genes mirror the proteins that are found to be associated with corrosion-mediated ALTR and are chemokine (C-X-C) motif ligand 9 also known as MIG or CXCL9, chemokine (C-X-C) motif ligand 10 also known as IP10 or CXCL10, interferon gamma or IFNγ, interleukin 6 or IL6, and interleukin 8 or IL8 as well as CD3.

In addition, twenty other genes listed in Table 2 were found to highly expressed or up-regulated in patients with corrosion-mediated ALTR as compared to patients with osteolysis-associated ALTR, and twenty-two genes listed in Table 3 were found to be up-regulated in patients with osteolysis-associated ALTR as compared to patients with corrosion-mediated ALTR. Also, in Table 4, six functional categories of genes up-regulated in patient with corrosion-mediated ALTR are listed. Any of these genes can be used in the methods and assays discussed herein to diagnoses, screen for, identify, predict, monitor, and/or treat corrosion-mediated ALTR. Preferred genes that can be used in the methods and assays are chemokines, genes related to cellular metal ion homeostasis, and cytokines and cytokine receptors (see Table 4).

By using these biomarkers, alone or preferably in conjunction, important predictions and determinations can be made regarding the risk of developing and/or the development of ALTR. In a preferred embodiment, the levels of expression of one or more or all of these genes is measured in the subject receiving the implant prior to the implantation, during the implantation or shortly thereafter, and a reference value of baseline gene expression level is obtained for the subject. In that manner, the level of expression of one or more genes measured after the implantation can be compared to the reference value, and any increase can be utilized in both choice of therapy as well as the determination for the amount and timing needed for monitoring by a health care provider, often prior to the onset of any noticeable symptoms.

In one embodiment, the expression of CXCL9 and/or CXCL10 is tested for in a subject. In another embodiment, the expression of IFNγ is tested for in a subject, and in another embodiment, the expression of IL6 and/or IL8 is tested for in a subject. In a preferred embodiment, the presence of CXCL9, CXCL10 and IFNγ is tested for in a subject. In a further preferred embodiment, the expression of all five genes is tested for in a subject.

Additionally, any one of the additional differentially regulated genes found in Table 2 or any combination of more than one up to all 20 differentially expressed genes can be used in the methods and assays of the present invention.

Additionally, any one of the differentially regulated genes found in Table 3 or any combination of more than one up to all 22 differentially expressed genes can be used in the methods and assays of the present invention.

Additionally, the expression of genes that encode chitotriosidase (CHIT1) and chemokine (C-C motif) ligand 18 (CCL18), associated with osteolysis-associated ALTR, can also be tested in conjunction with the expression of the genes associated with corrosion-mediated ALTR. In this manner, the specific subtype of ALTR, i.e., corrosion-mediated or osteolysis-associated, can be properly identified, diagnosed, monitored and/or treated.

Additionally, any one of the genes listed in Table 3 or any combination of more than one up to all 22 differentially expressed genes can be used for this purpose as well, as these are genes that are up-regulated in patients with osteolysis-associated ALTR as compared to patients with corrosion-mediated ALTR.

These biomarkers, in addition to being useful for clinicians to predict disease activity, are also useful as targets for therapy, for testing for developing therapies and research tools, such as testing materials used in implants for the activation of these genes.

Assays and Methods to Detect and Measure Genes Correlated with ALTR

In order to detect any of these transcripts or genes, a sample of biological tissue or fluid from a subject is obtained and prepared and analyzed for the presence of the genes. This can be achieved in numerous ways, by a diagnostic laboratory, and/or a health care provider.

Most methods start with obtaining a sample of biological tissue or bodily fluid from the subject and extracting, isolating and/or purifying the nucleic acid (e.g., genomic DNA, cDNA, RNA) from the tissue or fluid.

The nucleic acid can be obtained from any biological tissue. Preferred biological tissues include, but are not limited to, soft tissue surrounding the hip or implant or other joint including but not limited to periprosthetic pseudocapsule, bursal synovium, and adjacent skeletal muscle.

The nucleic acid can be obtained from any bodily fluid. Preferred fluids include, but are not limited to, synovial fluid surrounding the hip or implant or other joint, urine, blood, plasma and serum.

The nucleic acid is extracted, isolated and purified from the cells of the tissue or fluid by methods known in the art.

Once prepared, mRNA or other nucleic acids are analyzed by methods known to those of skill in the art. The nucleic acid sequence corresponding to a gene can be any length, with the understanding that longer sequences are more specific. Preferably a nucleic acid corresponding to a gene is at least 20 nucleotides in length. Preferred ranges are from 20 to 100 nucleotides in length, with from 30 to 60 nucleotides being more preferred, and from 40 to 50 being most preferred.

A preferred method of the invention is performing gene expression profiling of the sample. Gene expression profiling refers to examining expression of one or more RNAs in a cell, preferably mRNA. Often at least or up to 10, 100, 100, 10,000 or more different mRNAs are examined in a single experiment.

Methods for examining gene expression, are often hybridization based, and include, microarrays; RNA sequencing; Southern blots; Northern blots; dot blots; primer extension; nuclease protection; subtractive hybridization and isolation of non-duplexed molecules using, for example, hydroxyapatite; solution hybridization; filter hybridization; amplification techniques such as RT-PCR, and other PCR-related techniques, such as PCR with melting curve analysis, and PCR with mass spectrometry; fingerprinting, such as with restriction endonucleases; and the use of structure specific endonucleases. mRNA expression can also be analyzed using mass spectrometry techniques (e.g., MALDI or SELDI), liquid chromatography, and capillary gel electrophoresis. Any additional method known in the art can be used to detect the presence or absence of the transcripts. For a general description of these techniques, see also Sambrook et al. 1989; Kriegler 1990; and Ausebel et al. 1990.

The preferred method for the detection of the transcripts is the use of arrays or microarrays. These terms are used interchangeably and refer to any ordered arrangement on a surface or substrate of different molecules, referred to herein as “probes.” Each different probe of any array is capable of specifically recognizing and/or binding to a particular molecule, which is referred to herein as its “target” in the context of arrays. Examples of typical target molecules that can be detected using microarrays include mRNA transcripts, cRNA molecules, cDNA, PCR products, and proteins.

Microarrays are useful for simultaneously detecting the presence, absence and quantity of a plurality of different target molecules in a sample. The presence and quantity, or absence, of the probe's target molecule in a sample may be readily determined by analyzing whether and how much of a target has bound to a probe at a particular location on the surface or substrate.

In a preferred embodiment, arrays used in the present invention are “addressable arrays” where each different probe is associated with a particular “address.”

The arrays used in the present invention are preferable nucleic acid arrays that comprise a plurality of nucleic acid probes immobilized on a surface or substrate. The different nucleic acid probes are complementary to, and therefore can hybridize to, different target nucleic acid molecules in a sample. Thus, each probe can be used to simultaneously detect the presence and quantity of a plurality of different genes, e.g., the presence and abundance of different mRNA molecules, or of nucleic acid molecules derived therefrom (for example, cDNA or cRNA).

The arrays are preferably reproducible, allowing multiple copies of a given array to be produced and the results from each easily compared to one another. Preferably microarrays are small, and made from materials that are stable under binding conditions. A given binding site or unique set of binding sites in the microarray will specifically bind to the target. It will be appreciated that when cDNA complementary to the RNA of a cell is made and hybridized to a microarray under suitable conditions, the level or degree of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, the site on the array corresponding to a gene (i.e., capable of specifically binding a nucleic acid product of the gene) that is not transcribed in the cell will have little or no signal, while a gene for which mRNA is highly prevalent will have a relatively strong signal.

By way of example, GeneChip® (Affymetrix, Santa Clara, Calif.), generates data for the assessment of gene expression profiles and other biological assays. Oligonucleotide expression arrays simultaneously and quantitatively “interrogate” thousands of mRNA transcripts. Each transcript can be represented on a probe array by multiple probe pairs to differentiate among closely related members of gene families. Each probe contains millions of copies of a specific oligonucleotide probe, permitting the accurate and sensitive detection of even low-intensity mRNA hybridization patterns. After hybridization data is captured, using a scanner or optical detection systems, software can be used to automatically calculate the intensity values for each probe cell. Probe cell intensities can be used to calculate an average intensity for each gene, which correlates with mRNA abundance levels. Expression data can be quickly sorted based on any analysis parameter and displayed in a variety of graphical formats for any selected subset of genes.

Further examples of microarrays that can be used in the assays and methods of the invention are microarrays synthesized in accordance with techniques sometimes referred to as VLSIPS™ (Very Large Scale Immobilized Polymer Synthesis) technologies as described, for example, in U.S. Pat. Nos. 5,324,633; 5,744,305; 5,451,683; 5,482,867; 5,491,074; 5,624,711; 5,795,716; 5,831,070; 5,856,101; 5,858,659; 5,874,219; 5,968,740; 5,974,164; 5,981,185; 5,981,956; 6,025,601; 6,033,860; 6,090,555; 6,136,269; 6,022,963; 6,083,697; 6,291,183; 6,309,831; 6,416,949; 6,428,752 and 6,482,591.

Other exemplary arrays that are useful for use in the invention include, but are not limited to, Sentrix® Array or Sentrix® BeadChip Array available from Illumina®, Inc. (San Diego, Calif.) or others including beads in wells such as those described in U.S. Pat. Nos. 6,266,459; 6,355,431; 6,770,441; and 6,859,570. Arrays that have particles on the surface can also be used and include those described in U.S. Pat. Nos. 6,489,606; 7,106,513; 7,126,755; and 7,164,533.

An array of beads in a fluid format, such as a fluid stream of a flow cytometer or similar device, can also be used in methods for the invention. Exemplary formats that can be used in the invention to distinguish beads in a fluid sample using microfluidic devices are described, for example, in U.S. Pat. No. 6,524,793. Commercially available fluid formats for distinguishing beads include, for example, those used in XMAP™ technologies from Luminex or MPSS™ methods from Lynx Therapeutics.

A spotted microarray can also be used in a method of the invention. An exemplary spotted microarray is a CodeLink™ Array available from Amersham Biosciences.

Another microarray that is useful in the invention is one that is manufactured using inkjet printing methods such as SurePrint™ Technology available from Agilent Technologies. Other microarrays that can be used in the invention include, without limitation, those described in U.S. Pat. Nos. 5,429,807; 5,436,327; 5,561,071; 5,583,211; 5,658,734; 5,837,858; 5,919,523; 6,287,768; 6,287,776; 6,288,220; 6,297,006; 6,291,193; and 6,514,751.

DASL can be used for quantitative measurements of RNA target sequences as well as for DNA target sequences. DASL is described, for example, in Fan et al. (2004).

Additional techniques for rapid gene sequencing and analysis of gene expression include, SAGE (serial analysis of gene expression). For SAGE, a short sequence tag (typically about 10-14 bp) contains sufficient information to uniquely identify a transcript. These sequence tags can be linked together to form long serial molecules that can be cloned and sequenced. Quantitation of the number of times a particular tag is observed proves the expression level of the corresponding transcript (see, e.g., Velculescu et al. (1995); Velculescu et al. (1997)).

Screening and diagnostic method of the current invention may involve the amplification of the target loci. A preferred method for target amplification of nucleic acid sequences is using polymerases, in particular polymerase chain reaction (PCR). PCR or other polymerase-driven amplification methods obtain millions of copies of the relevant nucleic acid sequences which then can be used as substrates for probes or sequenced or used in other assays.

Amplification using polymerase chain reaction is particularly useful in the embodiments of the current invention. PCR is a rapid and versatile in vitro method for amplifying defined target DNA sequences present within a source of DNA. Usually, the method is designed to permit selective amplification of a specific target DNA sequence(s) within a heterogeneous collection of DNA sequences (e.g. total genomic DNA or a complex cDNA population). Mutation detection using the 5′→3′ exonuclease activity of Taq DNA polymerase (TaqMan™ assay) can also be used as a screening and diagnostic method of the current invention. Such an assay involves hybridization of three primers, the third primer being intended to bind just downstream of one of the conventional primers which should be allele-specific. The additional primer carries a blocking group at the 3′ terminal nucleotide so that it cannot prime new DNA synthesis and at its 5′ end carries a labeled group. In modern versions of the assay, the label is a fluorogenic group and the third primer also carries a quencher group. If the upstream primer which is bound to the same strand is able to prime successfully, Taq DNA polymerase will extend a new DNA strand until it encounters the third primer in which case its 5′→3′ exonuclease will degrade the primer causing release of separate nucleotides containing the dye and the quencher, and an observable increase in fluorescence.

PCR with melting curve analysis can also be used. PCR with melting curve analysis is an extension of PCR where the fluorescence is monitored over time as the temperature changes. Duplexes melt as the temperature increases and the hybridization of both PCR products and probes can be monitored. The temperature-dependent dissociation between two DNA-strands can be measured using a DNA-intercalating fluorophore such as SYBR green, EvaGreen or fluorophore-labelled DNA probes.

PCR with mass spectrometry uses mass spectrometry to detect the end product. Primer pairs are used and tagged with molecules of known masses, known as MassCodes. If DNA from any of the agent of primer panel is present, it will be amplified. Each amplified product will early its specific Masscodes. The PCR product is then purified to remove unbound primers, dNTPs, enzyme and other impurities. Finally, the purified PCR products are subject of ultraviolet as the chemical bond with nucleic acid and primers are photolabile. As the Masscodes are liberated from PCR products they are detected with a mass spectrometer.

When a probe is to be used to detect the presence of nucleic acids, the nucleic acid to be analyzed and the probe are incubated under conditions which promote stable hybrid formation of the target sequence in the probe and the target sequence in the nucleic acid. The desired stringency of the hybridization will depend on factors such as the uniqueness of the probe in the part of the genome being targeted, and can be altered by washing procedure, temperature, probe length and other conditions known in the art, as set forth in Sambrook et al. 1989.

Labeled probes are used to detect the hybrid, or alternatively, the probe is bound to a ligand which labeled either directly or indirectly. Suitable labels and methods for labeling are known in the art, and include biotin, fluorescence, chemiluminescence, enzymes, and radioactivity.

Assays using such probes include Southern blot, Northern blot analysis, and dot-blot hybridization, following procedures known in the art (e.g., Sambrook et al. (1989)).

RNA and DNA sequencing may also be used to detect expression of any of the genes.

### Proteins Correlated to ALTR

As shown in the examples, there is an increase in certain proteins in patients with corrosion-mediated ALTR. Thus, these proteins can be specifically used to diagnose, screen for, identify, predict, monitor and/or treat corrosion-mediated ALTR in a subject who has had a THA or another joint implant, or who is at risk for ALTR. These proteins are two chemokines, MIG or CXCL9 and IP10 or CXCL10, interferon gamma or INFγ, interleukin 6 or IL6, and interleukin 8 or IL8.

By using these biomarkers, alone or preferably in conjunction, important predictions and determinations can be made regarding the risk of developing and/or the development of ALTR. In a preferred embodiment, the amount or level of one or more or all of these proteins is measured in the subject prior to, during, or shortly after, implantation of the implant. In that manner, the amount or level of protein or proteins measured after the implantation in the subject can be compared to the amount or level of protein or proteins measured in the subject prior to, during, or shortly after, implantation. Alternatively, the amount of protein in the sample can be compared to a reference value. For example, the amounts of CXCL9, CXCL10, IL6 and IL8 in subjects with osteolysis-associated ALTR and those with corrosion-mediated ALTR are listed in Table 1. Any increase in these biomarkers can be utilized in both choice of therapy as well as the determination for the amount and timing needed for monitoring by a health care provider, prior to the onset of any noticeable symptoms.

In one embodiment, the level of CXCL9 and/or CXCL10 is tested for in a subject. In another embodiment, the presence of INFγ is tested for in a subject, and in another embodiment, the level of IL6 and/or IL8 is tested for in a subject. In a preferred embodiment, the level of CXCL9, CXCL10 and INFγ is tested for in a subject. In a further preferred embodiment, the level of all five proteins is tested for in a subject.

Additionally, the level of proteins, chitotriosidase (CHIT1) and chemokine (C-C motif) ligand 18 (CCL18), associated with osteolysis-associated ALTR, can also be tested in conjunction with the level of proteins associated with corrosion-mediated ALTR. In this manner, the specific subtype of ALTR, i.e., corrosion-mediated or osteolysis-associated, can be properly identified, diagnosed, monitored and/or treated.

These biomarkers, in addition to being useful for clinicians to predict disease activity, are also useful as targets for therapy, for testing for developing therapies and research tools, such as testing materials used in THA and other implants for the activation of these proteins.

### Assays and Methods to Detect Proteins Correlated to ALTR

A sample of biological tissue or bodily fluid from a subject is obtained. This can be achieved in numerous ways, by a diagnostic laboratory, and/or a health care provider.

The protein sample can be obtained from any biological tissue. Preferred biological tissues include, but are not limited to, soft tissue surrounding the hip or THA implant or other joint implant including but not limited to periprosthetic pseudocapsule, bursal synovium, and adjacent skeletal muscle.

The protein sample can be obtained from any bodily fluid. Preferred fluids include, but are not limited to, synovial fluid surrounding the hip or THA implant or other joint implant, urine, blood, plasma, and serum.

Protein is isolated and/or purified from the sample using any method known in the art, including but not limited to immunoaffinity chromatography.

Any method known in the art can be used for detecting and measuring increase levels of the proteins in a protein sample, but preferred methods include quantitative Western blot, immunoblot, quantitative mass spectrometry, enzyme-linked immunosorbent assays (ELISAs), radioimmunoassays (RIA), immunoradiometric assays (IRMA), and immunoenzymatic assays (IEMA) and sandwich assays using monoclonal and polyclonal antibodies.

Antibodies are a preferred method of detecting and measuring the inflammatory proteins in a sample. Such antibodies are available commercially or can be made by conventional methods known in the art. Such antibodies can be monoclonal or polyclonal and fragments thereof, and immunologic binding equivalents thereof. The term “antibody” means both a homologous molecular entity as well as a mixture, such as a serum product made up of several homologous molecular entities.

In a preferred embodiment, such antibodies will immunoprecipitate the proteins from a solution as well as react with proteins on a Western blot, immunoblot, ELISA, and other assays listed above.

Antibodies for use in these assays can be labeled covalently or non-covalently with an agent that provides a detectable signal. Any label and conjugation method known in the art can be used. Labels, include but are not limited to, enzymes, fluorescent agents, radiolabels, substrates, inhibitors, cofactors, magnetic particles, and chemiluminescent agents.

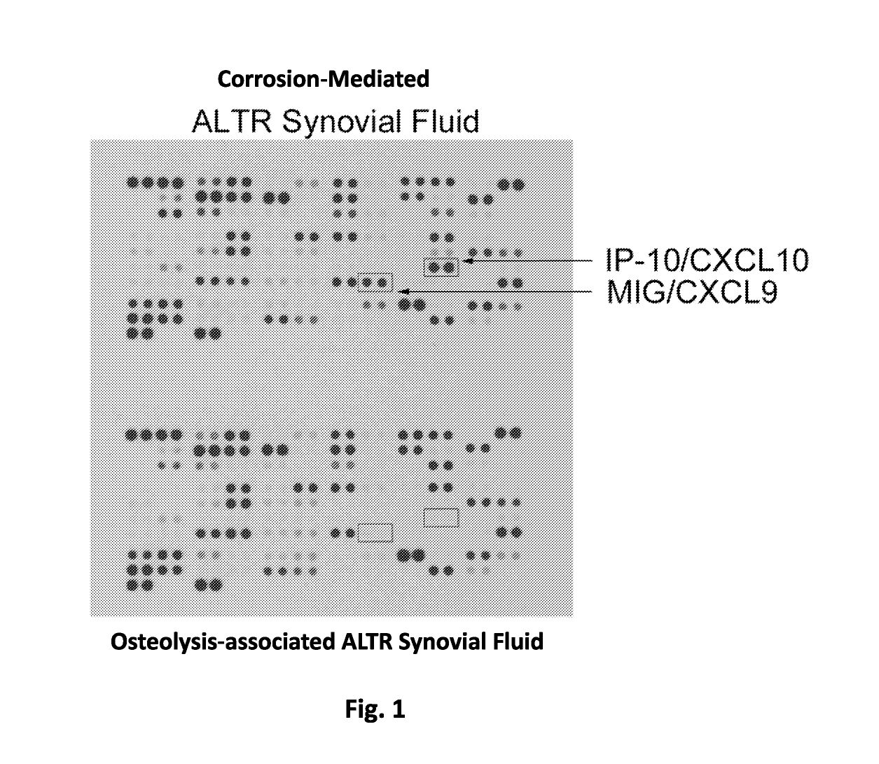

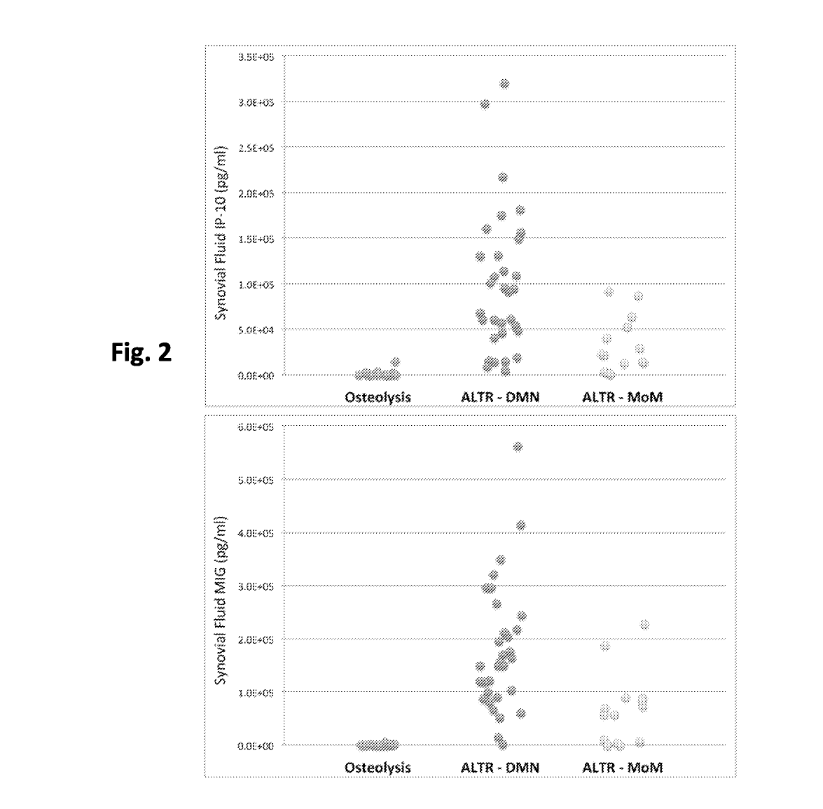

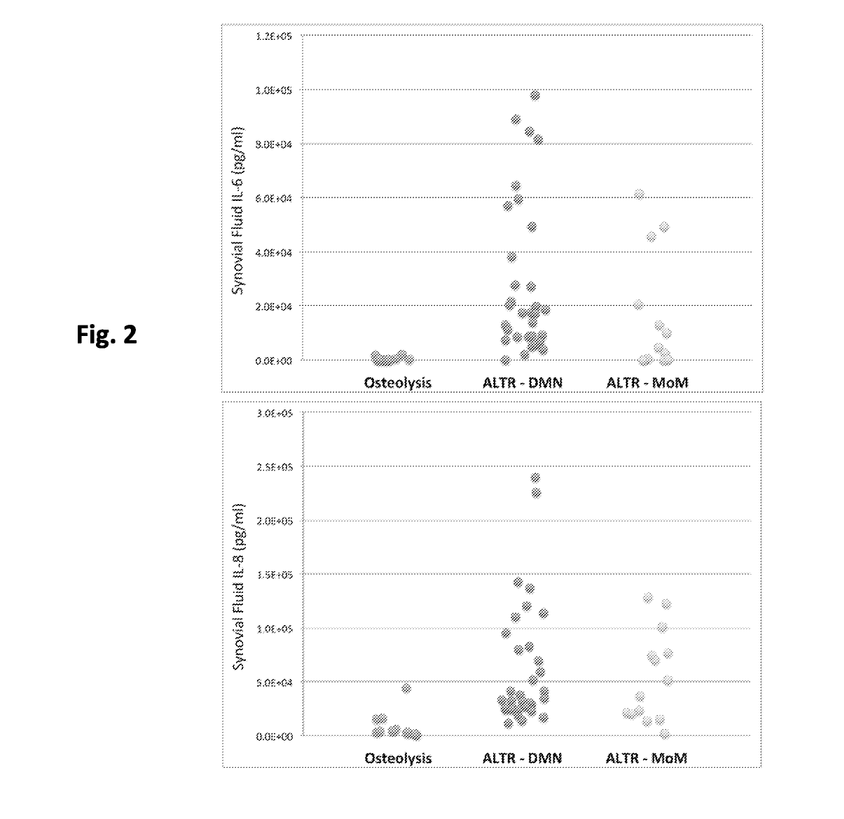

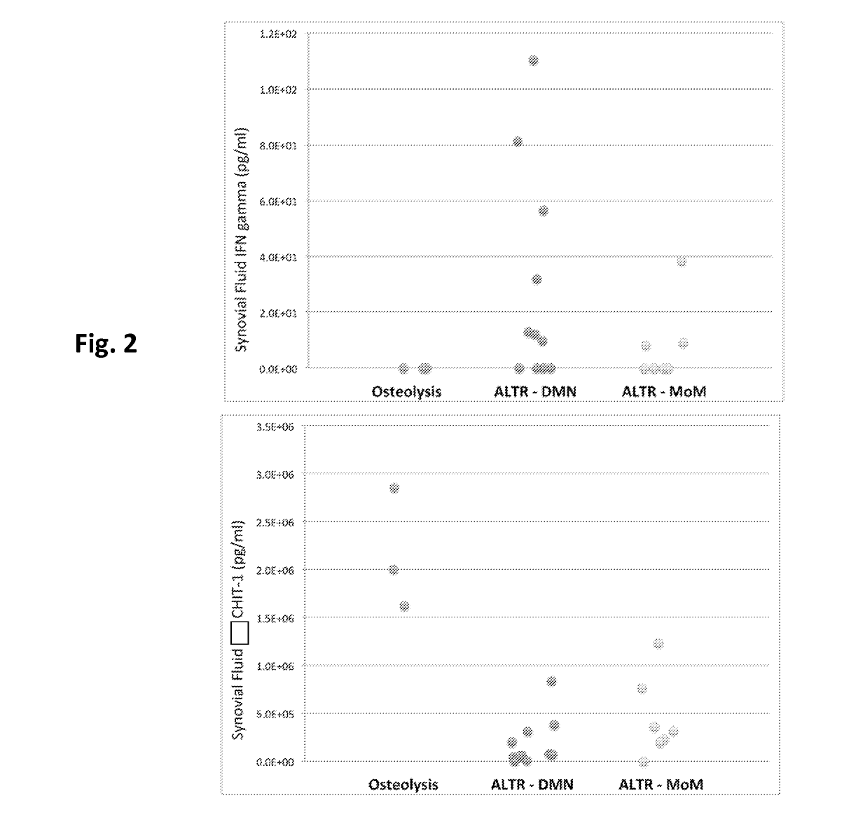

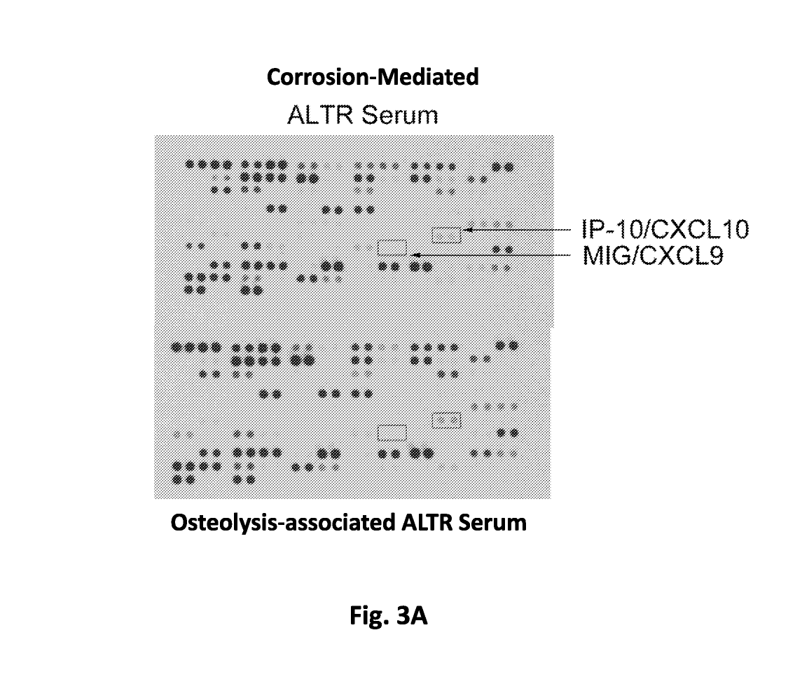

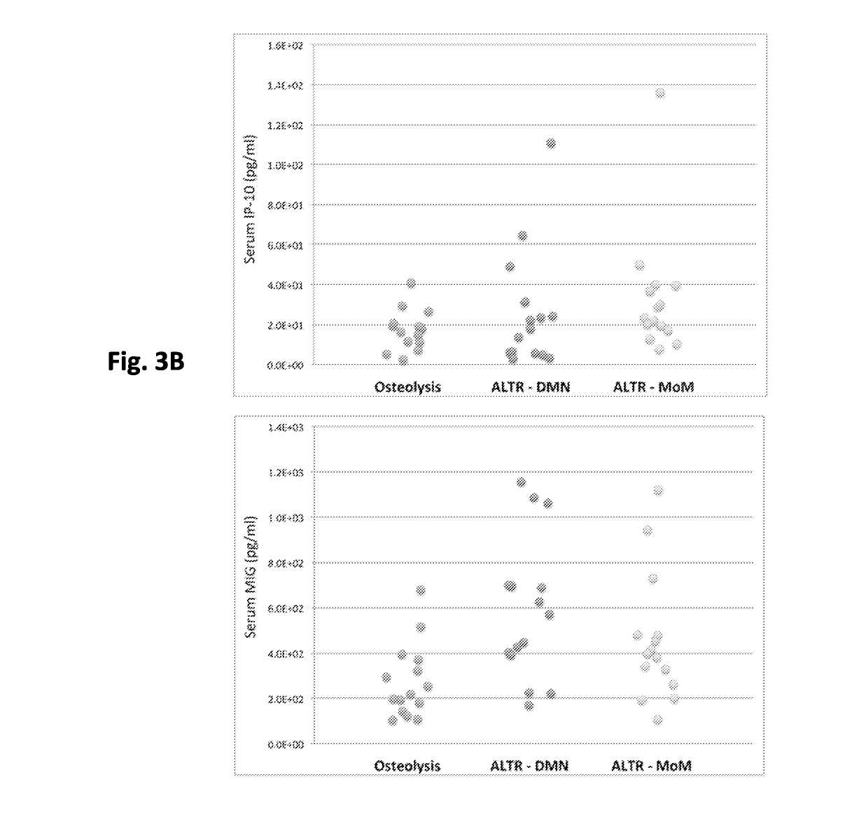

Assays for detection of the proteins are exemplified below in Examples 1 and 2, and include antibody arrays and ELISAs. As can be seen in FIGS. 1 and 3, which illustrate the use of an antibody array, CXCL9 and CXCL10 can be seen qualitatively on the array. In Table 1, the amounts of CXCL9, CXCL10, IL6, and IL8 protein in subject with corrosion-mediated ALTR, as measured by ELISA, are about five to 100 times greater than subjects with osteolysis-associated ALTR. FIG. 2 shows ELISA results for CXCL9, CXCL10, IL6, IL8, IFN gamma, and CHIT1 in patients with both osteolysis-associated ALTR, and corrosion-mediated ALTR.

### Treatment of ALTR and Prevention of Implant Failure

A further embodiment of the present invention is a method of treating corrosion-mediated ALTR and the prevention of the subsequent failure of the implant caused by the corrosion-mediated ALTR by decreasing, preventing or blocking the activation, amount and/or activity of CXCL9 and/or CXCL10 and/or IFNγ and/or IL6 and/or IL8, or by decreasing or preventing or blocking the expression of the CXCL9 and/or CXCL10 and/or IFNγ and/or IL6 and/or IL8 gene.

One such a method comprises administering to a subject in need thereof a therapeutically effective amount of an agent that decreases, prevents or blocks the activation, amount, or activity of CXCL9 and/or CXCL10 and/or IFNγ and/or IL6 and/or IL8, or an agent that decreases, prevents or blocks the expression of the CXCL9 and/or CXCL10 and/or IFNγ and/or IL6 and/or IL8 gene.

Such agents include but are not limited to chemicals, phytochemicals, pharmaceuticals, biologics, small organic molecules, antibodies, nucleic acids, peptides, and proteins.

Because interferon gamma activates CXCL9 and CXCL10, an agent that decreases IFNγ can be used in the method. These agents would include, but are not limited to, antibodies to IFNγ including, but not limited to, humanized monoclonal antibodies to IFNγ, such as fontolizumab, and human monoclonal antibodies, such as AMG811. Other agents include but are not limited to a combination of ribovarin and interferon alpha.

Antibodies to CXCL9, CXCL10, IL6 and IL8 can also be used in the method.

Antagonists to the receptor chemokine receptor 3, or CXCR3, to which CXCL9 and CXCL10, bind can also be used. This receptor was cloned and characterized by Loetscher in 1996. Antagonists include, but are not limited to, antibodies, and small molecular weight inhibitors, including the (aza)quinazolinone class of CXCR3 antagonists, such as AMG487 (see e.g., Wijtmans et al. (2008); Chen et al. (2012); Liu et al. (2009); Wijtmans et al. (2011)). Other inhibitors include, but are not limited to, fused pyrimidine derivatives (WO02/083143); N-1R-[3-(4-ethoxy-phenyl)-4-oxo-3,4-dihydro-pyrido[2,3-d]pyrimidin-2-yl]-ethyl-N-pyridin-3-ylmethyl-2-(4-fluoro-3-trifluoromethyl-phenyl)-acetamide (NBI-74330) (Heise et al. (2005)); 3-phenyl-3H-quinazolin-4-ones (Storelli et al. (2005)); 1-aryl-3-piperidin-4-yl-urea derivatives (Allen et al. (2007)); 5-(piperidin-4-yl)amino-1,2,4-thiadiazole derivatives (Watson et al. (2007)); compounds with a benzimidazole core and tethered acetophenone moiety (Hayes et al. (2008)); imidazo-pyrazine derivatives (Du et al. (2009)); and compounds with a pyrazinyl-piperazinyl-piperidine scaffold (Kim et al. (2011)).

Inhibiting the activity of the proteins can also be accomplished using “decoy” molecules which mimic the region of a target molecule any of the proteins binds and activates, such as CXCR3. Inhibition can also be effected by the use of a “dominantly interfering” molecule, or one in which the binding portion of proteins is retained but the molecule is truncated so that the activating domain is lacking. These molecules would bind to receptors in the pathway but be unproductive and block the receptors from binding to the activating molecule.

RNA interference or RNAi refers to the ability of double stranded RNA (dsRNA) to suppress the expression of a specific gene of interest in a homology-dependent manner. RNA interference commonly involves the use of dsRNAs that are greater than 500 bp, however, it can also be mediated through small interfering RNAs (siRNAs) or small hairpin RNAs (shRNAs), which can be 10 or more nucleotides in length and are typically greater than 18 nucleotides in length, around 20-25 base pairs in length.

These RNAi can also be used to down-regulate the expression of CXCL9, CXCL10, INFγ, IL6 and IL8, and can designed by methods known in the art using the sequences.

MicroRNA can also be used to down-regulate gene expression. MicroRNAs are small non-coding RNAs averaging 22 nucleotides that regulate the expression of their target mRNA transcripts by binding (Ambros (2004); Bartel (2009)). Binding of microRNAs to their targets is specified by complementary base pairing between positions 2-8 of the microRNA and the target 3′ untranslated region (3′ UTR), an mRNA component that influences translation, stability and localization (Bartel (2009)). Again microRNAs that bind to the 3′UTR of the mRNA of CXCL9, CXCL10, INFγ, IL6 and IL8 can designed by methods known in the art using the sequence of the mRNA.

All of these small molecules can be manufactured by methods known in the art.

A further embodiment of the present invention is a method of treating osteolysis-associated ALTR and the prevention of the subsequent failure of the implant caused by the osteolysis-associated ALTR. Previous studies by the inventors have demonstrated a key role of the inflammasome in mediating the cellular response and tissue reaction to polyethylene wear products.

One such method comprises administering to a subject in need thereof a therapeutically effective amount of an agent that decreases, prevents or blocks the activation, or activity of the inflammasome. Agents that inhibit the inflammasome have been reported and include, but are not limited to, a small molecule andrographolibe (Andro) (Gua et al. (2014)), lactate (Hogue et al. (2014)), polyenylpyrrole derivatives (Hua et al. (2013)), luteoloside (Fan et al. (2014)), as well as antioxidants, glyburide, probenecid, and anti-Ill therapy (Castejon and Pelegrin (2012)).

Any of the agents outlined above, i.e., antibodies, small molecules, RNAi, also can be used in these methods of decreasing, preventing or blocking the activation or activity of the inflammasome, for treating osteolysis-associated ALTR.

A further embodiment of the present invention comprises administering to a subject in need thereof a therapeutically effective amount of an agent that decreases, prevents or blocks the activation, amount, or activity of CHIT1 and/or CCL18, or an agent that decreases, prevents or blocks the expression of the CHIT1 and/or CCL18 gene.

Any of the agents outlined above, i.e., antibodies, small molecules, RNAi, can be used in these methods for treating osteolysis-associated ALTR.

A further embodiment of the present invention is a method that blocks the activity of osteoclasts that are the bone cell type that mediates the osteolytic process associated with osteolysis-associated ALTR. A commercially available antibody to RANK ligand (denosumab), effectively blocks the formation and activation of osteoclasts and osteoclast-mediated osteolysis and can be used to treat osteolysis-associated ALTR.

Additional agents, including bisphosphonates that are widely used in the treatment of bone loss associated with osteoporosis and other forms of pathologic bone loss, also can be used to directly block osteolysis-associated ALTR.

The current invention provides methods for the use of the biomarkers for early detection of osteolysis-associated ALTR prior to the development of clinically significant osteolysis and loss of implant fixation. This will permit the institution of preventative therapies targeting the inflammatory reaction to polyethylene wear products that cause osteolysis-associated ALTR at an earlier stage than was possible before based upon the use of the biomarkers.

The current invention also provides methods for providing treatment for both subtypes of ALTR at an earlier stage than was possible before based upon the use of the biomarkers. In this manner, not only is a subject definitively diagnosed with ALTR, they can also be definitively diagnosed with the subtype of ALTR, i.e., osteolysis-associated or corrosion-mediated, and can be treated more effectively with agents that target the specific subtype of ALTR and subsequent implant failure can be prevented. Using the biomarkers provided herein for the first time allows clinicians and health care providers to definitely diagnose and treat ALTR prior to implant failure and the need for revision surgery.

Alternatively, a clinician or health care provider may order other more expensive and intrusive diagnostic tests, such as imaging with MRI or x-rays, for a subject after testing with the biomarkers indicates ALTR. Also, in the alternative, if the subject after testing has increased amounts of one or more protein biomarkers, and/or increased expression of one or more gene biomarkers, a clinician or health care provider may decide to monitor the subject and test for the biomarkers again at a set time, before ordering more expensive and intrusive diagnostic tests or prescribing treatment.

### Kits

It is contemplated that all of the assays disclosed herein can be in kit form for use by a health care provider and/or a diagnostic laboratory.

Assays for the detection and quantitation of one or more of the genes can be incorporated into kits. Such kits would include probes for one or more of the genes, reagents for isolating and purifying nucleic acids from biological tissue or bodily fluid, reagents for performing assays on the isolated and purified nucleic acid, instructions for use, and reference values or the means or instructions for obtaining reference values in a control sample for the included genes.

A preferred kit would include probes for CXCL9 and/or CXCL10 and/or IFNγ and/or IL6 and/or IL8. In another embodiment of the present invention, the kit would include probes for CHIT1 and/or CCL18.

A preferred embodiment of these kits would have the probes attached to a solid state. A most preferred embodiment would have the probes in a microarray format wherein nucleic acid probes for the genes would be in an ordered arrangement on a surface or substrate.

In a further embodiment, a kit would include reagents for testing for CXCL9 and/or CXCL10 and/or IFNγ and/or IL6 and/or IL8 proteins, Such a kit could include antibodies that recognize the peptide or peptides of interest, reagents for isolating and/or purifying protein from a biological tissue or bodily fluid, reagents for performing assays on the isolated and purified protein, instructions for use, and reference values or the means or instructions for obtaining reference values for the quantity or level of peptides in a control sample.

In another embodiment of the present invention, the kit would reagents for testing for CHIT1 and/or CCL18, including antibodies that recognize these proteins.

In another preferred embodiment of the kits would have antibodies to the proteins attached to a solid state in an ordered arrangement.

### Drug Screening Assays and Research Tools

All of the biomarkers disclosed herein can be used as the basis for drug screening assays and research tools.

In one embodiment, a protein chosen from the group consisting of CXCL9, CXCL10, IFNγ, IL6 and IL8, or a protein encoded by any one of the genes listed in Tables 2 and 4, can be used in drug screening assays, free in solution, or affixed to a solid support. In another embodiment, CHIT1 and CCL18, or a protein encoded by any one of the genes listed in Table 3, can be used in drug screening assays. All of these forms can be used in binding assays to determine if agents being tested form complexes with the peptides, proteins or fragments, or if the agent being tested interferes with the formation of a complex between the peptide or protein and a known ligand.

Thus, the present invention provides for methods and assays for screening agents for treatment of corrosion-mediated ALTR, comprising contacting or incubating the test agent with a encoding the protein chosen from the group consisting of CXCL9, CXCL10, IFNγ, IL6 and IL8, or a protein encoded by any one of the genes listed in Tables 2 and 4, and detecting the presence of a complex between the protein and the agent or the presence of a complex between the protein and a ligand, by methods known in the art. In such competitive binding assays, the polypeptide or fragment is typically labeled. Free polypeptide is separated form that in the complex, and the amount of free or uncomplexed polypeptide is measured. This measurement indicates the amount of binding of the test agent to the polypeptide or its interference with the binding of the polypeptide to a ligand.

The same methods can be used to screen agents for the treatment of osteolysis-associated ALTR using CHIT1, CCL18, or a protein encoded by any of the genes listed in Table 3.

High throughput screening can also be used to screen for therapeutic agents. Small peptides or molecules can be synthesized and bound to a surface and contacted with the polypeptides encoded by the gene signature transcripts, and washed. The bound peptide is visualized and detected by methods known in the art.

Antibodies to the polypeptides can also be used in competitive drug screening assays. The antibodies compete with the agent being tested for binding to the polypeptides. The antibodies can be used to find agents that have antigenic determinants on the polypeptides, which in turn can be used to develop monoclonal antibodies that target the active sites of the polypeptides.

The invention also provides for polypeptides to be used for rational drug design where structural analogs of biologically active polypeptides can be designed. Such analogs would interfere with the polypeptide in vivo, such as by non-productive binding to target. In this approach the three-dimensional structure of the protein is determined by any method known in the art including but not limited to x-ray crystallography, and computer modeling. Information can also be obtained using the structure of homologous proteins or target-specific antibodies.

Using these techniques, agents can be designed which act as inhibitors or antagonists of the polypeptides, or act as decoys, binding to target molecules non-productively and blocking binding of the active polypeptide.

A further embodiment of the present invention is gene constructs comprising any one of the differentially expressed transcripts and a vector. These gene construct can be used for testing of therapeutic agents as well as basic research regarding ALTR. These gene constructs can also be used to transform host cells can be transformed by methods known in the art.

The resulting transformed cells can be used for testing for therapeutic agents as well as basic research regarding ALTR. Specifically, cells can be transformed with any one of the differentially expressed transcripts, and contacted with a test agent. The resulting expression of the transcript can be detected and compared to the expression of the transcript in the cell before contact with the agent.

The expression of the transcripts in host cells can be detected and measured by any method known in the art, including but not limited to, reporter gene assays.

The gene constructs as well as the host cells transformed with these gene constructs can also be the basis for transgenic animals for testing both as research tools and for therapeutic agents. Such animals would include but are not limited to, nude mice. Phenotypes can be correlated to the genes and looked at in order to determine the genes effect on the animals as well as the change in phenotype after administration or contact with a potential therapeutic agent.

The gene constructs and host cells transformed with the gene constructs can also be administered to murine models of ALTR, for analyzing test agents as well as basic research and to test potential materials for implants.

### EXAMPLES

The present invention may be better understood by reference to the following non-limiting examples, which are presented in order to more fully illustrate the preferred embodiments of the invention. They should in no way be construed to limit the broad scope of the invention.

### Example 1

### Materials and Methods for Identification of Synovial Fluid

### Protein Components and Gene Expression Profiles

### Patients

Patients with the diagnosis of corrosion-mediated ALTR or osteolysis-associated ALTR were identified retrospectively from the Osteolysis Tissue Registry and Repository at Hospital for Special Surgery. This prospective database collects demographics, selected clinical data, periprosthetic tissue, and fluids from all consenting patients undergoing revision THA. Two groups of corrosion-mediated ALTR patients were selected: the Dual-Modular Neck group (DMN) had a MoP or CoP bearing surface with a dual-modular neck (cobalt-chromium-molybdenum, CoCrMo) and TMZF (titanium, molybdenum, zirconia, iron) stem (Stryker, Rejuvenate), and the MoM THA group had a MoM bearing surface (CoCrMo) with a metallic sleeve adapter at the head-neck junction and titanium stem (Smith & Nephew, Birmingham THA). For comparative purposes, a group of patients with MoP bearing surfaces and a diagnosis of osteolysis-associated ALTR were also selected. All studies were approved by the Institutional Review Board (IRB) of Hospital for Special Surgery.

### Collection and Processing of Tissues and Fluids

Serum and periprosthetic tissue was collected from patients undergoing revision surgery at Hospital for Special Surgery for suspected ALTR. Serum was prepared by standard methods and aliquoted and immediately stored at −80° C. Areas of tissue containing viable cellular components were identified by H&E staining of frozen sections, and immediately thereafter subaliquoted and either submerged in RNAlater (AMbion) and stored at −80° C. to maintain RNA integrity, or subjected to routine formalin fixation and paraffin embedding for histopathological analysis. Where possible, synovial fluid was also obtained, centrifuged at 1500rpm for 10 minutes to remove cellular components, aliquoted and stored at −80° C. For comparative purposes, similar samples were obtained from patients undergoing hip revision surgery for periprosthetic osteolysis secondary to polyethylene wear, and patients receiving index surgery for degenerative arthritis.

### Antibody Arrays

Proteome Profiler™ Human XL Cytokine Array Kits (Catalog #ARY022, R&D Systems, Minneapolis, Minn.) were used in accordance with the manufacturer's recommendations. For each array, 50 μl of synovial fluid was used for hybridization. In some experiments, Proteome Profiler™ Human Chemokine Array Kits (Catalog #ARY017) and Proteome Profiler™ Human Cytokine Array Kits (Catalog #ARY005) were utilized.

### ELISA

The following ELISA kits were purchased and used in accordance with recommendations: OptEIA™ Human IL6 ELISA set (Catalog #555220), OptEIA™ Human IL8 ELISA set (Catalog #555244), OptEIA™ Human IP10 ELISA set (Catalog #550926), OptEIA™ Human MIG ELISA set (Catalog #5509980), OptEIA™ Human IFNγ/ELISA set (Catalog #555142) (all OptEIA™ ELISA sets from BD Bioscience, Franklin Lakes, N.J.), and CircuLex Human Chitotriosidase ELISA Kit (MBL International, Woburn, Mass.). Synovial fluid and serum samples were diluted appropriately to allow detection within the linear range for each assay. Standards were included on every assay plate and used for quantitation.

### RNA Isolation

Aliquots of minced peri-implant tissue in RNAlater (see above) were thawed on ice and the tissue was immediately transferred to Trizol (Invitrogen) and homogenized on ice using a handheld Polytron homogenizer. Total RNA was then purified, precipitated and washed in accordance with the recommended Trizol protocol. Some samples were further purified using RNAeasy columns (Qiagen) as recommended. Samples were quantitated using a Nanodrop 2000 spectrophotometer and subsequently subjected to BioAnalyzer analysis to determine RNA integrity. Samples with a RIN in excess of 7.0 were approved for microarray analysis and real time polymerase chain reaction.

### Microarray Analysis

One microgram total RNA was used to synthesize cRNA using the Affymetrix expression protocol (Expression Analysis Technical Manual; Affymetrix, Santa Clara, Calif.). Ten micrograms labeled and fragmented cRNA was hybridized to Affymetrix U133 2.0 chips (Affymetrix, Santa Clara, Calif.), and detection signal was calculated using MAS5 algorithm in GCOS (Expression Analysis Technical Manual; Affymetrix) and sample normalization was performed using global scaling.

Fold change differences and p-values were calculated using ANOVA, and genes were considered differentially regulated if they had a fold change of ±2 between groups, and if the false-discovery-rate corrected p-value was <0.05. Hierarchical clustering was performed with signal values on differentially-regulated genes, normalized to between 0 and 1 for each gene, and then clustered via Ward's method.

### Real Time Polymerase Chain Reaction

500 ng aliquots of total cellular RNA were reverse transcribed using oligo-dT primers and MMLV reverse transcriptase (First Strand eDNA Synthesis Kit, Fermentas) as recommended by the manufacturer. Real time quantitative PCR (qPCR) was carried out in duplicate using the iCycler iQ thermal cycler detection system (Bio-Rad Laboratories Inc., Hercules, Calif.). Reactions included iQ™ SYBR® Green Supermix reagent (Bio-Rad Laboratories Inc., Hercules, Calif.), 10 ng cDNA, and forward and reverse primers each at a concentration of 250 nM in a total volume of 25 μl, mRNA amounts were normalized relative to the house-keeping genes HPRT or GAPDH, and quantified using the ΔΔCt method 49. Generation of only the correct amplification products was confirmed using melting point curve analysis of the products. The sequences of the oligonucleotide primers used were:

GADPH
SEQ ID NO: 1
ATCAAGAAGGTGGTGAAGCA;
SEQ ID NO: 2
GTCGCTGTTGAAGTCAGAGGA;
CHIT1
SEQ ID NO: 3
GAGAGTGGTGCAGCAGCCA;
SEQ ID NO: 4
CCACTGTTGCACAGCAGCAT;
MIG
SEQ ID NO: 5
TTGGGCATCATCTTGCTGGTTCT;
SEQ ID NO: 6
TGGCTGACCTGTTTCTCCCACTT;
IFNγ
SEQ ID NO: 7
GACTTCGAAAAGCTGACTAAT
SEQ ID NO: 8
TTGGATGAGTTCATGTATTGC
I16
SEQ ID NO: 9
GGAGACTTGCCTGGTAA
SEQ ID NO: 10
GCATTTGTGGTTGGGTCA
IL8
SEQ ID NO: 11
CTGCAGCTCTGTGTGAAGGTG
SEQ ID NO: 12
TAAGTTCTTTAGCACTCCTTGGCAA
CD3
SEQ ID NO: 13
ATGGAGTTCGCCAGTCGAGA
SEQ ID NO: 14
CATTGGGCAACAGAGTCTGCT

### Example 2

### Cytokine and Chemokine Levels in Synovial Fluid and Serum

The cytokine and chemokine antibody arrays described in Example 1 were used to provide an overview of levels of these critical inflammatory mediators in synovial fluid from ALTR patients and controls. As illustrated in FIG. 1, synovial fluid from corrosion-mediated ALTR was highly similar to that from osteolysis-associated ALTR. However, amongst the 10⁷ chemokines and cytokines represented on these arrays, 2 chemokines, MIG/CXCL9 and IP10/CXCL10, were present in notably higher levels in the corrosion-mediated ALTR fluid.

Additional array analysis (data not shown) revealed that in addition to these chemokines, IL6 and IL8 were found at elevated levels in synovial fluid from corrosion-mediated ALTR and osteolysis-associated ALTR patients.

To extend and quantitate these findings, levels of IL6, IL8, MIG and IP10 in synovial fluid from larger numbers of patients were analyzed by ELISA as described in Example 1. As shown in FIG. 2, levels of MIG and IP10 were significantly and substantially higher in corrosion-mediated ALTR patients compared to osteolysis-associated ALTR, the latter of which were within the reported normal range (Table 1). IL6 and IL8 were also significantly higher in corrosion-mediated ALTR patients than in osteolysis-associated ALTR (FIG. 2 and Table 1).

Since MIG and IP10 are strongly induced in response to interferon gamma, the levels of interferon gamma were measured in the synovial fluid samples. Detectable levels of interferon gamma were found in several of the corrosion-mediated ALTR samples but in none of the osteolysis-associated ALTR samples. See FIG. 2 and Table 1.

As also shown in FIG. 2, the levels of CHIT1 in synovial fluid from patients with osteolysis-mediated ALTR was significantly higher than the levels in patients with corrosion-mediated ALTR.

Antibody array analysis of serum samples revealed similar profiles for corrosion-mediated ALTR and osteolysis-associated ALTR (FIG. 3A). ELISA analysis confirmed that the variations in serum levels of MIG and IP10 between groups (FIG. 3B and Table1)

Taken together, these results indicate that the interferon responsive chemokines MIG/CXCL9 and IP10/CXCL10 were dramatically elevated in the synovial fluid of THR patients experiencing corrosion-mediated ALTR associated with Metal-on-Metal or Modular Neck design implants compared to patients experiencing osteolysis-associated ALTR secondary to polyethylene wear, where levels of CHIT1 protein were elevated. A similar pattern was seen in the serum. Other inflammatory modulators, including IL6 and IL8, were elevated in both subtypes of ALTR, but more so in corrosion-mediated ALTR.

TABLE 1
Levels of Selected Proteins in the Synovial Fluid and Serum of Patients ALTR

| | | | Corrosion- | | Corrosion- | |
|---|---|---|---|---|---|---|
| | | Osteolysis | mediated | p-value | mediated | p-value |
| | Protein | ALTR | ALTR-DMN | (DMN) | ALTR-MOM | (MoM) |
| Synovial | MIG | 1,320 (1,901) | 178,442 (120,107) | 8.46 E−6 | 68,609 (68,867) | 2.52 E−3 |
| Fluid | IP10 | 1,880 (4,304) | 100,137 (78,158) | 9.32 E−5 | 32,829 (30,575) | 2.00 E−3 |
| | IL6 | 497 (786) | 28,583 (28,481) | 1.53 E−3 | 16,102 (21,768) | 2.10 E−2 |
| | IL8 | 8,621 (12,820) | 64,172 (58,125) | 2.21 E−3 | 54,338 (41,930) | 1.32 E−3 |
| Serum | MIG | 273 (163) | 592 (315) | 1.68 E−3 | 456 (279) | 0.036 |
| | IP10 | 17.4 (9.8) | 25.7 (29.5) | 0.31 | 32.9 (31.0) | 0.074 |

Data represented as mean (standard deviation) for all samples.
Amounts are in pg/ml.
P-values are for comparison to the osteolysis group.

### Example 3

### Analysis of Periprosthetic Gene Expression Profiles

To complement and extend the above measurements of cytokines and chemokines in the fluids of ALTR patients, the gene expression profiles in the corresponding periprosthetic tissues of selected patients was analyzed. As detailed above, care was given to choosing tissues with evidence of ongoing ALTR for RNA extraction. A total of 8 DMN corrosion-mediated ALTR RNA samples were of sufficient quality for microarray analysis, and were compared with 8 MoP osteolysis-associated ALTR RNA samples (previously reported in Koulouvaris et al. (2007)). All 16 samples were analyzed using Affymetrix U133 2.0 microarrays in the same core laboratory as described in Example 1. The data were processed using Genespring GX12 (Agilent) to identify genes significantly differentially expressed between the two ALTR groups.

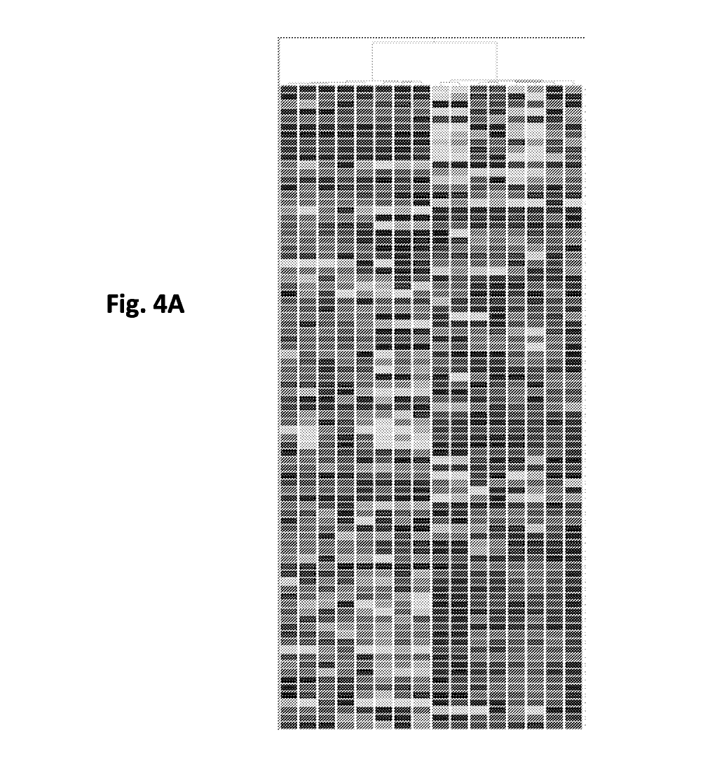

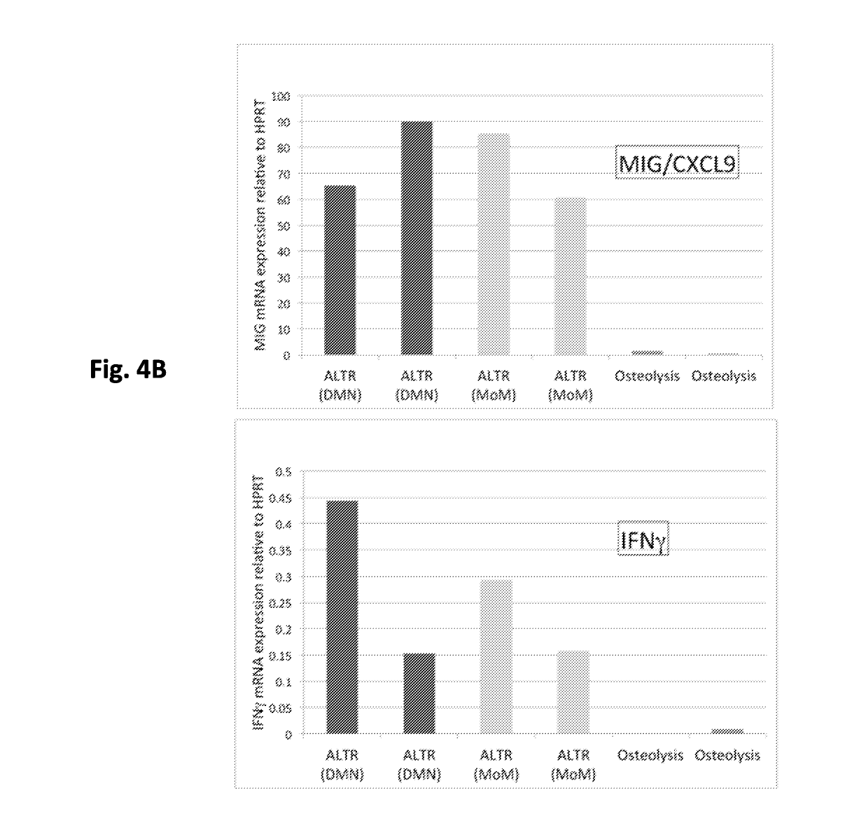

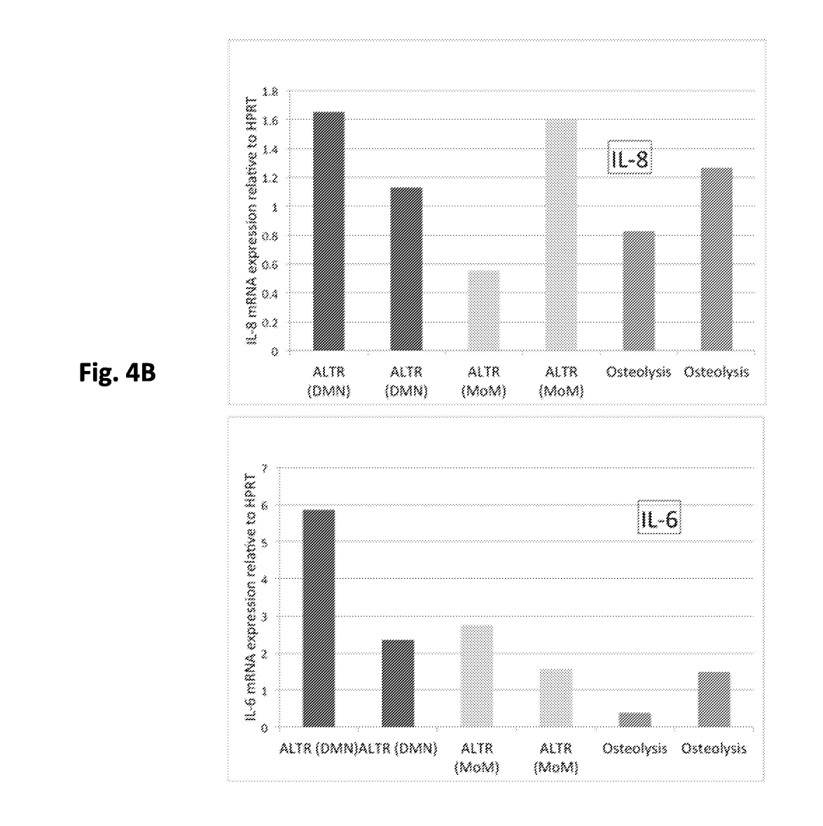

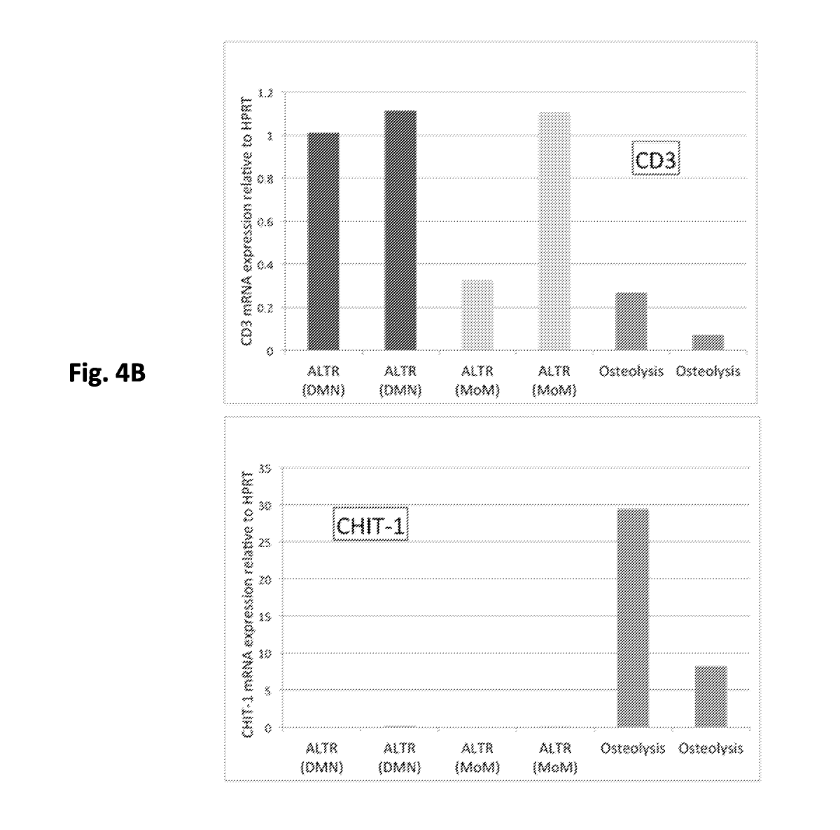

Hierarchical clustering analysis of genes with a 5-fold or greater difference between groups revealed clear differences between the expression profiles of patients with corrosion-mediated ALTR and osteolysis-associated ALTR, as shown in FIG. 4A. In this figure, the eight columns on the right represent patients with corrosion-mediated ALTR and the eight columns on the left represent patients with osteolysis-associated ALTR. Each row is a gene. As can be seen by the figure, there are marked areas of difference (i.e., dark and light patterns) between the eight columns on the right and the eight on the left.

Array analysis of MoM corrosion-mediated ALTR samples were highly similar to the DMN corrosion-mediated ALTR samples, and clustered together in hierarchical clustering analysis (data not shown).

Amongst the most prominently up-regulated genes in corrosion-mediated ALTR were numerous chemokines including MIG/CXCL9 and IP-10/CXCL10, mirroring the highly elevated levels of these proteins identified in the synovial fluid of corrosion-mediated ALTR patients (Table 2), whereas CHIT1 was, as expected, amongst the genes most highly up-regulated in osteolysis-associated ALTR as compared to corrosion-mediated ALTR (Table 3).

Real-time PCR validated the overexpression of MIG/CXCL9, IFNγ, IL8, and IL6 mRNAs in corrosion-mediated ALTR (both from DMN and MoM) and the overexpression of CHIT1 in osteolysis-associated ALTR, and extended the array data to show elevated CD3 expression in corrosion-mediated ALTR, reflecting the lymphocytic infiltration in these tissues (FIG. 4B).

Finally, pathway analysis of the microarray data (DAVID: Database for Annotation, Visualization, and Integrated Discovery, Huang et al. (2009a); Huang et al. (2009b)) showed that immune system activation, including lymphocyte activation pathways and signaling by chemokines/cytokines and their receptors were greatly amplified in corrosion-mediated ALTR, consistent with the histopathology, and analysis of protein and RNA markers. Interestingly, corrosion-mediated ALTR also showed enrichment of the cellular metal ion homeostasis pathway, suggesting possible direct involvement of implant derived wear in the tissue response (Table 4).

TABLE 2
Genes Most Highly Expressed in Tissue from Corrosion-Mediated ALTR from
DMN Group as Compared to Tissue from Osteolysis-Associated ALTR Tissue

| Probe Set ID | FC | Gene Symbol | Gene Title |
|---|---|---|---|
| 203915_at | 40.04 | CXCL9 | chemokine (C-X-C motif) ligand 9 |
| 204533_at | 30.95 | CXCL10 | chemokine (C-X-C motif) ligand 10 |
| 204580_at | 12.63 | MMP12 | matrix metallopeptidase 12 (macrophage elastase) |
| 211796_s_at | 11.98 | TRBC1 /// | T cell receptor beta constant 1 /// T cell receptor beta |
| | | TRBC2 | constant 2 |
| 210072_at | 11.93 | CCL19 | chemokine (C-C motif) ligand 19 |
| 210915_x_at | 11.21 | TRBC1 | T cell receptor beta constant 1 |
| 206407_s_at | 11.14 | CCL13 | chemokine (C-C motif) ligand 13 |
| 214038_at | 10.72 | CCL8 | chemokine (C-C motif) ligand 8 |
| 206134_at | 10.34 | ADAMDEC1 | ADAM-like, decysin 1 |
| 205242_at | 8.96 | CXCL13 | chemokine (C-X-C motif) ligand 13 |
| 226218_at | 8.73 | IL7R | interleukin 7 receptor |
| 201445_at | 8.58 | CNN3 | calponin 3, acidic |
| 205569_at | 8.55 | LAMP3 | lysosomal-associated membrane protein 3 |
| 1555745_a_at | 8.44 | LYZ | lysozyme |
| 211430_s_at | 8.29 | IGHG1 | immunoglobulin heavy constant gamma 1 (G1m marker) |
| 209795_at | 8.06 | CD69 | CD69 molecule |
| 211339_s_at | 7.95 | ITK | IL2-inducible T-cell kinase |
| 202270_at | 7.50 | GBP1 | guanylate binding protein 1, interferon-inducible, 67 kDa |
| 213193_x_at | 7.30 | TRBC1 | T cell receptor beta constant 1 |
| 201110_s_at | 7.29 | THBS1 | thrombospondin 1 |
| 204891_s_at | 7.25 | LCK | lymphocyte-specific protein tyrosine kinase |
| 238581_at | 7.20 | GBP5 | guanylate binding protein 5 |

FC—fold change

TABLE 3
Genes Most Down-Regulated in Tissue from Corrosion-Mediated ALTR from
DMN Group as Compared to Tissue from Osteolysis-Associated ALTR Tissue

| Probe Set ID | FC | Gene Symbo [symbol] | Gene Title |
|---|---|---|---|
| 219697_at | 19.17 | HS3ST2 | heparan sulfate (glucosamine) 3-O-sulfotransferase 2 |
| 208168_s_at | 15.09 | CHIT1 | chitinase 1 (chitotriosidase) |
| 206007_at | 14.97 | PRG4 | proteoglycan 4 |
| 215856_at | 11.99 | SIGLEC15 | sialic acid binding Ig-like lectin 15 |
| 1553706_at | 11.50 | HTRA4 | HtrA serine peptidase 4 |
| 227556_at | 10.36 | NME7 | non-metastatic cells 7, protein expressed in |
| | | | (nucleoside-diphosphate kinase) |
| 207857_at | 8.43 | LILRA2 | leukocyte immunoglobulin-like receptor, subfamily A |
| | | | (with TM domain), member 2 |
| 1557197_a_at | 8.41 | LGALS3 | lectin, galactoside-binding, soluble, 3 |
| 213956_at | 8.30 | CEP350 | centrosomal protein 350 kDa |
| 229797_at | 7.94 | MCOLN3 | mucolipin 3 |
| 208212_s_at | 7.83 | ALK | anaplastic lymphoma receptor tyrosine kinase |
| 203979_at | 7.73 | CYP27A1 | cytochrome P450, family 27, subfamily A, polypeptide 1 |
| 227404_s_at | 7.66 | EGR1 | Early growth response 1 |
| 219525_at | 7.60 | SLC47A1 | solute carrier family 47, member 1 |
| 213229_at | 7.45 | DICER1 | dicer 1, ribonuclease type III |
| 229070_at | 7.28 | C6orf105 | chromosome 6 open reading frame 105 |
| 218211_s_at | 7.10 | MLPH | melanophilin |
| 230690_at | 6.98 | TUBB1 | tubulin, beta 1 |
| 238706_at | 6.83 | PAPD4 | PAP associated domain containing 4 |
| 235019_at | 6.81 | CPM | carboxypeptidase M |
| 215123_at | 6.80 | NPIPL3 | nuclear pore complex interacting protein-like 3 |
| 1556336_at | 6.75 | RBMX | RNA binding motif protein, X-linked |

FC—fold change [symbol]
indicates data missing or illegible when filed

TABLE 4
Functional Analysis of Selected Pathways Up-regulated Genes in Corrosion-
Mediated ALTR Tissues as Compared to Osteolysis-Associated ALTR Tissues

| Pathway | p-value | Genes |
|---|---|---|
| Immune response | 1.49E−26 | IGHG1, IGHG2, IGLV1-44, CD8A, IGHM, CXCL12, CXCL10, |
| | | SH2D1A, CXCR4, IL1B, FCGR3A, FCGR3B, LTB, ICAM1, |
| | | POU2AF1, GBP5, IGL@, GZMA, TRBC1, HLA-DQA2, HLA-DQA1, |
| | | IGKV1-5, CST7, CCR2, CTSC, GBP4, CLEC5A, GBP1, ITGAL, |
| | | YWHAZ, CCL2, ENPP2, CXCL9, CCL8, CCL5, IL7R, CLEC10A, |
| | | CCL4, CCL7, GCH1, THBS1, IGKC, CD27, IL6, THEMIS, CCL19, |
| | | PSMB9, TNFSF10, CYBB, CCL13, CXCL13, P2RY14, CD274, |
| | | IGHV4-31, RNF19B, IFI6 |
| Lymphocyte | 9.16E−13 | ITGAL, ICAM1, CD3D, IKZF1, CD8A, THEMIS, IL21R, FKBP1A, |
| activation | | ITGA4, IL7R, ITGB1, CXCL12, SLAMF1, CTNNB1, NLRC3, |
| | | CXCR4, BCL11B, LCK, CD2, IRF1, IRF4 |
| Chemokine | 1.92E−10 | CCL13, CCL2, CXCL13, CXCL9, CCL8, CCL19, CCL5, CCL4, |
| activity | | CXCL12, CCL7, CXCL10 |
| Cytokine/ | 8.66E−10 | IL2RB, IL6, CCL2, IL21R, CXCL9, CCL19, CCL8, IL7R, CCL5, |
| Cytokine Receptor | | CXCL12, CCL4, CCL7, CXCL10, INHBA, CCL13, TNFSF10, |
| | | CXCR4, CXCL13, CCR2, IL1B, IL13RA1, LTB, CD27 |
| Inflammatory | 3.48E−08 | ITGAL, YWHAZ, IL6, CCL2, LYZ, CXCL9, CCL19, CCL8, CCL5, |
| response | | CCL4, CCL7, CXCL10, CYBB, CCL13, CXCR4, CXCL13, CCR2, |
| | | IL1B, SERPINA1, THBS1 |
| Cellular metal ion | 1.38E−06 | CCL2, CCL19, FKBP1A, CCL5, CXCL12, CCL7, CD38, APP, |
| homeostasis | | CCL13, CXCL13, CXCR4, LCK, CCR2, IL1B |

### REFERENCES

Allen et al. (2007) Bioorg. Med. Chem Letters 17:697-701
Ambros (2004) Nature 431:350
Amstutz and Grigoris (1996) Clin. Orthop. Relat. Res. 329(Suppl):S11-S34
Ausebel et al. 1990 Current Protocols in Molecular Biology
Bartel (2009) Cell 136:215
Bosker et al. (2012) J. Bone Joint Surg. Br. 94:755-61
Campbell et al. (2010) Clin. Orthop. Relat. Res 468:2321-27
Castejon and Pelegrin (2012) Expert Opinion Invest. Drugs 21:995-1007
Chana et al. (2012) J. Bone Joint Surg. Br. 94:281-86
Chen et al, (2012) Bioorg. Med Chem. Letters 22:357-62
Cooper et al. (2013) J. Bone Joint Surg. Am. 95:865-72
Cooper et al. (2012) J. Bone Joint Surg. Am. 94:1655-61
Davies and Willert (2005) J. Bone Joint Surg. Am. 87:18-27
Du et al. (2009) Bioorg. Med. Chem. Letters 19:5200-04
Dyrkacz et al. (2003) J. Arthroplasty 28:1036-40
Fabi et al. (2012) Orthopedics 35:e1009-16
Fan et al. (2004) Genome Res. 14:878-85
Fan et al. (2014) PlosOne 9:e89961
Fricka et al. (2012) J. Arthroplasty 27(8 Suppl):26-31.e1
Gallo et al. (2014) Acta Biomater. 10:2354-66
Gill et al. (2012) J Bone Joint Surg. Br. 94:895-900
Grammatopoulos et al. (2013) J. Bone Joint Surg. Am. 95:e81
Guo et al. (20140 Autophagy 10:972-85
Hart et al. (2012) J. Bone Joint Surg. Am. 94:317-25
Hayes et al. (2008) Bioorg. Med. Chem. Letters 18:1573-76
Heise et at (2005) J. Pharmacol. Exp. Ther. 313:1263-71
Hogue et al. (2014) Gastroenterolgy 146:1763-74
Hua et al. (2013) PlosOne 8:e7654
Huang et al. (2009a) Nature Protocols 4:44-57
Huang et al. (2009b) Nucleic Acids Research 37:1-13
Huber et al. (2009) Acta Biomater. 5:172-80
Kim et al. (2011) Bioorg. Med. Chem. Letters 21:6982-86.
Koulouvaris et al. (2007) J. Orthoped. Res. 26:109-16
Kop and Swarts (2009). J Arthroplasty 24:1019-23
Kriegler (1990) Gene Transfer and Expression: A Laboratory Manual
Liu et al. (2009) Bioorg. Med. Chem. Letters 19:5114-18
Loetscher et al, (1996) J. Exp. Med. 184:963-69
Lovey et al. (2008) Abstract, ORS
Mahendra et al. (2009) Acta Orthop. 80:653-59
Meyer et al. (2012) Clin. Orthop. Relat. Res. 470:3101-8
Mittal et al. (2013) PLoS One 8:e63470
Natu et al. (2012) J. Clin. Pathol. 65:409-418
Perino et al. (2014) BMC Clinical Pathology 14:39
Sambrook et al. (1989) Molecular Cloning, A Laboratory Manual
Schmalzried et al. (1992) J. Bone Joint Surg. Am. 74:849-63
Srinivasan et al. (2012) J. Am. Acad. Orthop. Surg. 20:214-22
Storelli et al. (2005) Bioorg. Med. Chem. Letters 15:2910-13
Watson et al. (2007) Bioorg. Med. Chem. Letters 17:6806-10
Werner et al. (2013) J. Arthroplasty 28:543.e13-5
Willert et al. (2005) J. Bone Joint Surg. Am. 87:28-36
Wijtmans et al. (2011) Bioorg. Med. Chem. Letters 19:3384-93
Wijtmans et al. (2008) Chem Med Chem 3:861-72
Velculescu et al. (1995) Science 270:484-7
Velculescu et al. (1997) Cell 88:243-51

1. A kit for diagnosing, screening for, identifying, predicting and monitoring adverse local tissue reactions comprising a microarray comprising one or more nucleic acid probes in an ordered arrangement, reagents for isolating and/or purifying nucleic acids from biological tissue or bodily fluid, reagents for performing assays on the isolated and/or purified nucleic acid, instructions for use, and reference values, or the means or instructions for obtaining reference values in a sample, wherein the nucleic acid probes recognize least seven genes comprising:
CXCL9, CXCL10, IFNγ, IL6, IL8, CHIT1 and CCL18.

2. (canceled)

3. A kit for diagnosing, screening for, identifying, predicting and monitoring adverse local tissue reaction comprising at least one antibody that recognizes at least seven proteins comprising:
CXCL9, CXCL10, IFNγ, IL6, IL8, CHIT1 and CCL18, reagents for isolating and/or purifying proteins from biological tissue or bodily fluid, reagents for performing assays on the isolated and/or purified proteins, instructions for use, and reference values, or the means or instructions for obtaining reference values in a sample.

4. (canceled)

5. (canceled)

6. The method of claim 13, wherein the subject is human.

7. The method of claim 13, wherein the joint is chosen from the group consisting of hip, knee, and shoulder.

8. The method of claim 13, wherein the biological tissue is chosen from the group consisting of periprosthetic pseudocapsule, bursal synovium, and adjacent skeletal muscle.

9. The method of claim 13, wherein the bodily fluid is chosen from the group consisting of the synovial fluid surrounding the implant or joint, urine, blood, plasma, and serum.

10. The method of claim 13, wherein the nucleic acid is mRNA, cDNA or genomic DNA.

11. The method of claim 13, wherein the level of expression of the genes is measured by an assay chosen from the group consisting of microarrays; Southern blots; Northern blots; dot blots; primer extension; nuclease protection; subtractive hybridization; solution hybridization; filter hybridization; polymerase chain reaction; polymerase chain reaction with melting curve analysis; polymerase chain reaction with mass spectrometry; fingerprinting; RNA sequencing; mass spectrometry techniques; liquid chromatography; and capillary gel electrophoresis

12. The method of claim 13, wherein the period of time that the method is performed after the subject has received the implant is about six months.

13. A method of diagnosing, screening for, identifying or predicting adverse local tissue reactions in a subject who has received an implant in a joint, comprising:
a. obtaining biological tissue or bodily fluid from the subject;
b. isolating and purifying a sample of nucleic acid from the biological tissue or bodily fluid;
c. measuring the level of expression of at least five genes chosen from the group consisting of CXCL9, CXCL10, IFNγ, IL6, and IL8;
d. comparing the level of expression of the genes obtained in step (c) with a reference value of the level of expression of the same genes, wherein the reference value is chosen from the group consisting of: (i) a predetermined value; (ii) the level of expression of the same genes in the subject prior to, at the time of, or shortly after, receiving the implant, wherein the level of expression of the genes is from a sample taken from the same bodily fluid or biological tissue and similarly processed; and (iii) the level of expression of the same genes in a control, wherein the level of expression of the genes is from a sample taken from the same bodily fluid or biological tissue and similarly processed and the control is not suffering from corrosion-mediated adverse local tissue reactions;
e. measuring the level of expression of at least two more genes chosen from the group consisting of CHIT1 and CCL18;
f. comparing the level of expression of the genes obtained in step (e) with a reference value of the level of expression of the same genes, wherein the reference value is chosen from the group consisting of: (i) a predetermined value; (ii) the level of expression of the same genes in the subject prior to, at the time of, or shortly after receiving the implant, wherein the level of expression of the genes is from a sample taken from the same bodily fluid or biological tissue and similarly processed; and (iii) the level of expression of the same genes in a control, wherein the level of expression of the genes is from a sample taken from the same bodily fluid or biological tissue and similarly processed and the control is not suffering from osteolysis-associated adverse local tissue reactions; and
g. diagnosing, identifying or predicting corrosion-mediated adverse local tissue reactions in a subject when the level of expression measured in step (c) is greater than the corresponding reference value, and diagnosing, identifying or predicting osteolysis-associated adverse local tissue reactions in a subject when the level of expression measured in step (e) is greater than the corresponding reference value,
wherein the method is performed at a period of time after the subject has received an implant to a joint.

14.-15. (canceled)

16. The method of claim 22, wherein the subject is human.

17. The method of claim 22, wherein the joint is chosen from the group consisting of hip, knee, and shoulder.

18. The method of claim 22, wherein the biological tissue is chosen from the group consisting of periprosthetic pseudocapsule, bursal synovium, and adjacent skeletal muscle.

19. The method of claim 22, wherein the bodily fluid is chosen from the group consisting of the synovial fluid surrounding the implant or joint, urine, blood, plasma, and serum.

20. The method of claim 22, wherein the level proteins is measured by an assay chosen from the group consisting of quantitative Western blot, immunoblot, quantitative mass spectrometry, enzyme-linked immunosorbent assays (ELISAs), radioimmunoassays (RIA), immunoradiometric assays (IRMA), and immunoenzymatic assays (IEMA) and sandwich assays.

21. The method of claim 22, wherein the period of time that the method is performed after the subject has received the implant is about six months.

22. A method of diagnosing, screening for, identifying or predicting adverse local tissue reactions in a subject who has received an implant in a joint, comprising:
a. obtaining biological tissue or bodily fluid from the subject;
b. isolating and purifying a sample of protein from the biological tissue or bodily fluid;
c. measuring the level of at least five proteins chosen from the group consisting of CXCL9, CXCL10, IFNγ, IL6, and IL8;
d. comparing the level of one or more proteins obtained in step (c) with a reference value of the level of the same protein or proteins wherein the reference value is chosen from the group consisting of: (i) a predetermined value; (ii) the level of the same protein or proteins in the subject prior to, at the time of, or shortly after receiving the implant, wherein of the level of the same protein or proteins is from a sample taken from the same bodily fluid or biological tissue and similarly processed; and (iii) the level of the same protein or proteins in a control, wherein the level of the same protein or proteins is from a sample taken from the same bodily fluid or biological tissue and similarly processed and the control is not suffering from corrosion-mediated adverse local tissue reactions;
e. measuring the level of at least two proteins chosen from the group consisting of CHIT1 and CCL18;
f. comparing the level of one or more proteins obtained in step (e) with a reference value of the level of the same protein or proteins wherein the reference value is chosen from the group consisting of: (i) a predetermined value; (ii) the level of the same protein or proteins in the subject prior to, at the time of, or shortly after receiving the implant, wherein of the level of the same protein or proteins is from a sample taken from the same bodily fluid or biological tissue and similarly processed; and (iii) the level of the same protein or proteins in a control, wherein the level of the same protein or proteins is from a sample taken from the same bodily fluid or biological tissue and similarly processed and the control is not suffering from osteolysis-associated adverse local tissue reactions; and
h. diagnosing, identifying or predicting corrosion-mediated adverse local tissue reaction in a subject when the level of protein or proteins measured in step (c) is greater than the corresponding reference value, and diagnosing, identifying or predicting osteolysis-associated adverse local tissue reactions in a subject when the level of expression measured in step (e) is greater than the corresponding reference value,
wherein the method is performed at a period of time after the subject has received an implant to a joint.

23.-41. (canceled)
